(19) United States
(12) Patent Application Publication
(10) Pub. No.: US 2015/0265572 A1
(43) Pub. Date: Sep. 24, 2015

(54) INJECTABLE CANCER COMPOSITIONS

(71) Applicant: GENSPERA, INC., San Antonio, TX (US)

(72) Inventors: Andrew X Chen, San Diego, CA (US); Yali Tsai, San Diego, CA (US)

(73) Assignee: GENSPERA, INC., San Antonio, TX (US)

(21) Appl. No.: 14/436,479

(22) PCT Filed: Oct. 14, 2013
(86) PCT No.: PCT/US13/64889

§ 371 Date: Apr. 16, 2015

Related U.S. Application Data
(60) Provisional application No. 61/714,662, filed on Oct. 16, 2012.

(51) Int. Cl. A61K 31/365; A61K 47/48; A61K 49/00; A61K 47/26; A61K 47/14; A61K 9/00; A61K 47/24

(52) U.S. Cl. CPC A61K 31/365; A61K 9/0019; A61K 47/48338; A61K 47/24; A61K 47/26; A61K 47/14; A61K 49/0004; A61K 47/48246

(57) ABSTRACT

Provided herein are therapeutic prodrug compositions which may be delivered to a patient via an injectable emulsion, comprising a therapeutic drug linked to a peptide that is efficiently and specifically cleaved by a selected protease associated with a cell proliferative disorder, including cancer cells, for example, prostate, liver or breast cancer cells, in a patient. Also provided herein are methods of treating cell proliferative disorders, including cancers, with the therapeutic prodrug compositions.

### PRIORITY

This application claims priority to U.S. Provisional Application No. 61/714,662, which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Oct. 14, 2013, is named GENS0004PCT_SL.txt and is 113,192 bytes in size.

### FIELD OF THE INVENTION

This invention relates generally to the targeted activation and delivery of therapeutic drugs to cells that produce selected proteins (i.e., proteases), or markers, for certain types of tumor-associated cells or cancer cells in a patient, for example, fibroblast activation protein (FAP), prostate specific membrane antigen (PSMA), prostate specific antigen (PSA) and human glandular kallikrein 2 (hK2). This invention also relates to injectable emulsion compositions which contain a prodrug that includes a therapeutic agent linked to an amino acid sequence containing a specific cleavage site for a selected protein, for example, FAP, PSMA, PSA and hK2. This invention also relates to methods and compositions for imaging subjects using an injectable emulsion composition which contains a prodrug that includes a therapeutic agent linked to an amino acid sequence containing a specific cleavage site for a selected protein.

### BACKGROUND OF THE INVENTION

Peptide prodrugs have been designed to deliver therapeutic drugs directly to cells associated with a cell proliferative disorder, tumor-associated cells (for example, tumor-associated endothelial and stromal cells), and cancer cells in a patient. These novel peptide prodrugs contain cleavage sites specifically designed to be cleaved by certain proteins that are expressed by cells associated with a cell proliferative disorder, cancer cells or cells associated with a tumor. For example, U.S. Pat. Nos. 7,906,477, 7,053,042 and 8,450,280 describe peptide prodrug compositions that are specifically cleaved by human glandular kallikrein 2 (hK2); U.S. Pat. Nos. 7,767,648 and 7,468,354 describe peptide prodrug compositions that are specifically cleaved by prostate specific membrane antigen (PSMA); U.S. Pat. Nos. 6,265,540; 6,504,014; 6,410,514; 6,545,131; and 7,635,682 describe peptide prodrug compositions that are specifically cleaved by prostate specific antigen (PSA); and U.S. patent application Ser. Nos. 12/087,398 and 13/471,316 describe peptide prodrug compositions that are specifically cleaved by fibroblast activation protein (FAP).

Therapeutic drugs that may be used in the peptide prodrugs described herein include certain therapeutic drugs which may contain primary amines (see, e.g., U.S. patent application Ser. No. 13/471,316). For example, the use of sesquiterpene-lactones such as thapsigargin and thapsigargicin, and their derivatives and analogues, as therapeutically active ingredients in peptide specific prodrugs for prostate and breast cancer has been described in multiple issued patents and patent applications. See, for example, U.S. Pat. Nos. 7,906,477, 7,767,648, and 6,545,131.

The prodrugs described herein also may be used in a combined approach to imaging, diagnosis and targeted treatment of cell proliferative disorders and cancers. U.S. patent application Ser. No. 13/257,131, for example, describes methods and compositions for imaging subjects using PSA-, PSMA- and hK2-specific peptide prodrugs.

The presently disclosed therapeutic drug compounds and prodrugs (e.g., peptide prodrugs) thereof, and their variations (for example, a prodrug with a detectable label or imaging compound), are sometimes referred to herein collectively as “active compounds.”

The active compounds described herein have limited solubility in water. Due to their limited solubility, an aqueous solution of the active compounds of the present invention requires a solubilizer to dissolve the active compound. Drug solubilizers commonly used in drug formulations include water-soluble/miscible organic solvents such as ethanol, surfactants such as polysorbate 80 or Cremophor, or cyclodextrins etc. However, these formulations are not desirable for injection-based delivery of the active compounds described herein as almost all these solubilizers are associated with certain toxicities including pain and irritation at the injection site, hypersensitivity or anaphylactic reactions. Thus, there is a need for injectable emulsion compositions that solubilize the active compounds of the present invention to the desired concentration without use of any water-soluble or toxic solubilizer.

The active compounds of the present invention are also unstable in water. In an aqueous environment, the active compounds of the present invention can degrade as much as about 12% or more in 7 days when stored at 60° C. For a formulation to be useful as a drug product, the degradation of the drug substance in the formulation must be less than about 10% within 1-2 years at a regular storage temperature such as room temperature (25-30° C.) or refrigerator temperature (2-8° C.).

Thus it is desirable to create an oil-in-water emulsion of the active compounds of the present invention such that the active compounds of the present invention may be stored for long periods of time and be provided to a patient in an injectable form.

The advantages of the present invention include ease of use. The injectable emulsion compositions of the invention may be stored and transported as a lyophilized product and re-suspended at the site of patient administration using water. Further, the injectable emulsion compositions described herein allow for the active compounds of the invention to be administered in a concentration suitable for injection, as opposed to an infusion. Additionally, the injectable emulsion compounds described herein may be stored and transported in a single ready-to-use vial to which only water must be added for administration, requiring no precise mixing or preparation of components on the part of the patient or administrator of the drug. Finally, the lyophilized formulations of the active compounds of the invention are chemically stable.

Other advantages of the present invention will be apparent to one of skill in the art based on the present disclosure.

### SUMMARY OF THE INVENTION

The present invention provides therapeutic prodrug compositions which may be delivered to a patient via an injectable emulsion, comprising a therapeutic drug linked to a peptide that is efficiently and specifically cleaved by a selected protease associated with a cell proliferative disorder and/or cancer cells, for example, prostate, liver or breast cancer cells, or tumor-associated cells in a patient. The linkage substantially inhibits the non-specific toxicity of the therapeutic drug, and cleavage of the peptide releases the drug, activating it or restoring its non-specific toxicity.

The present invention also provides therapeutic prodrugs (i.e., active compounds) in a safe and commercially feasible injectable emulsion that is (a) free of any water-soluble and toxic solubilizer, such as organic solvents or surfactants, (b) sufficiently stable to provide an acceptable shelf life, (c) composed of small oil droplets and filterable though a 0.2-micron filter, (d) transparent or translucent and (e) lyophilizable.

The invention also provides a method for treating cell proliferative disorders, including those involving the production of, for example, FAP, PSMA, PSA or hK2, in subjects having or at risk of having such disorders. The method involves administering to the subject a therapeutically effective amount of the active compounds of the invention. In this embodiment, the active compounds may be administered via injection.

In one aspect the invention features a peptide containing an amino acid sequence that includes a cleavage site specific for a selected protease, for example, FAP, PSMA, PSA or hK2, or an enzyme having a proteolytic activity of a selected protease. The peptides of the invention are preferably not more than 20 amino acids in length, more preferably not more than ten amino acids in length, and even more preferably about 6 amino acids in length, about 5 amino acids in length or about 4 amino acids in length. The amino acid sequences of the invention may be linear and/or may have side chain linkages.

In one embodiment, the peptides further comprise a capping group attached to the N-terminus of the peptide, wherein the capping group inhibits endopeptidase activity on the peptide. In some embodiments, the capping group is selected from the group consisting of acetyl, morpholinocarbonyl, benzyloxycarbonyl, glutaryl, and succinyl substituents. In another embodiment, the capping group comprises one or more of acetyl, morpholinocarbonyl, benzyloxycarbonyl, glutaryl or succinyl substituents.

Provided herein, according to one aspect are compositions in an injectable emulsion comprising a prodrug, the prodrug comprising a therapeutically active drug, and a peptide comprising an amino acid sequence having a cleavage site specific for an enzyme having a proteolytic activity of a selected protease, for example, FAP, PSMA, PSA, and hK2, wherein the peptide is 20 or fewer amino acids in length, and wherein the peptide is linked to the therapeutically active drug to inhibit the therapeutic activity of the drug, and wherein the therapeutically active drug is cleaved from the peptide upon proteolysis by an enzyme having a proteolytic activity of the selected protease.

In one embodiment, the therapeutically active drug has a primary amine. In one embodiment, the peptide is linked directly to the therapeutic drug. In another embodiment, the peptide is linked directly to a primary amine group on the drug.

In another embodiment, the peptide is linked to the therapeutic drug via a linker. In a related embodiment, the linker comprises one or more of an amino acid sequence, a primary amine or a carboxyl-containing alkyl, alkenyl or arginyl group.

In one embodiment, the therapeutically active drug is a sesquiterpene lactone.

In a variation of this embodiment, the therapeutically active drug is thapsigargin or a thapsigargin derivative. In a further variation of this embodiment, the thapsigargin or thapsigargin derivative contains a primary amine. In this embodiment, a preferred derivative is 8-O-(12-aminododecanoyl)-8-O-debutanoyl thapsigargin (12ADT). Further, in this embodiment, another preferred derivative is 8-O-(12-[L-leucinoylamino]dodecanoyl)-8-O-debutanoylthapsigargin (L12ADT).

In one embodiment, the therapeutically active drug inhibits a sarcoplasmic reticulum and endoplasmic reticulum Ca²⁺-ATPase (SERCA) pump.

In one embodiment, the therapeutically active drug has an LC₅₀ toward FAP-,

PSMA-, PSA-, or hK2-producing tissue of at most 20 μM. In a related embodiment, the therapeutically active drug has an LC₅₀ toward FAP-, PSMA-, PSA- or hK2-producing tissue of less than or equal to 2.0 μM.

Provided herein, according to one aspect of the invention is a method of producing a prodrug, the method comprising the step of linking a therapeutically active drug and a peptide comprising an amino acid sequence having a cleavage site specific for an enzyme having a proteolytic activity of a selected protease, for example, FAP, PSMA, PSA or hK2, wherein the peptide is 20 or fewer amino acids in length, and wherein the peptide is linked to the therapeutically active drug to inhibit the therapeutic activity of the drug, and wherein the therapeutically active drug is cleaved from the peptide upon proteolysis by an enzyme having a proteolytic activity of the selected protease.

Provided herein, according to one aspect are methods of treating a cell proliferative disorder, comprising administering the compositions described herein in a therapeutically effective amount to a subject having the cell proliferative disorder.

In one embodiment, the disorder is benign. In another embodiment, the disorder is malignant. In this embodiment, the malignant disorder may be an epithelial cancer. Further, in this embodiment, the malignant disorder may further be any epithelial cancer that expresses a protease that selectively cleaves an active compound of the invention (for example, PSA, hK2, FAP or PSMA), or any epithelial cancer that expresses a protease that selectively cleaves an active compound of the invention (for example, PSA, hK2, FAP or PSMA) in its vasculature.

In another embodiment, the malignant disorder is a sarcoma. For example, in this embodiment, the malignant tumor may be of cancerous bone, cartilage, fat, muscle, vascular, or hematopoietic tissues. In this embodiment, the malignant disorder may further be a sarcoma that expresses a protease that selectively cleaves an active compound of the invention (for example, PSA, hK2, FAP or PSMA).

In yet another embodiment, the disorder may be an inflammatory condition (for example, rheumatoid arthritis).

In one aspect of this embodiment of the invention, provided herein are methods of imaging selected protease-producing tissue, for example FAP-, PSMA-, PSA-, and hK2-producing tissue, the methods comprising: a) administering a peptide of the present invention linked to a lipophilic imaging label to a subject having or suspected of having a cell-proliferative disorder associated with the production of a selected protease; b) allowing a sufficient period of time to pass to allow cleavage of the peptide by the selected protease; c) allowing the lipophilic imaging label to accumulate in the tissue; d) allowing clearance of uncleaved peptide from the subject to provide a reliable imaging of the imaging label; and e) imaging the subject for diagnostic purposes.

In one further aspect of the invention, the method is a method of imaging soft tissue and/or bone metastases which produce a selected protease.

In another further aspect of the invention, an active compound of the invention may be used to image and diagnose a cell proliferative disorder or cancer. In this embodiment, a preferred derivative is 8-O-(12-aminododecanoyl)-8-O-debutanoyl thapsigargin (12ADT). In this embodiment, another preferred derivative is 8-O-(12-[L-leucinoylamino] do decanoyl)-8-O-debutanoylthapsigargin (L12ADT).

In a further aspect of the invention, a cell proliferative disorder is being imaged and targeted. In one alternative embodiment, the cell proliferative disorder is cancer. In a further aspect of this embodiment, the cancer being imaged and targeted is an epithelial cancer. In another aspect of the invention, the cell proliferative disorder may express PSA, PSMA, hK2 or FAP.

In another further aspect of this embodiment of the invention, the cancer being imaged and targeted is a sarcoma. In this aspect of the invention, the malignant disorder may further be a sarcoma that expresses PSA, PSMA, hK2 or FAP.

Further, the presently disclosed subject matter provides selective targeting of specific proteases, such as PSA, FAP, hK2 or PSMA, and makes use of the proteolytic activity of a protease to amplify an imaging signal.

### DRAWINGS

FIG. 1 is a portion of the amino acid sequence of Semenogelin I (SEQ ID NOs: 21-24) and Semenogelin IT (SEQ ID NOs: 25-31), showing the cleavage sites for human kallikrein 2.

FIG. 2 is a table (Table 1) showing amino acid sequences of peptides hydrolyzed by human glandular kallikrein2 (hK2) (SEQ ID NOS 144-157, respectively, in order of appearance). FIG. 2 discloses “NO₂-Y-G-K-A-X1-X2-X3-Dap-F-K(ABZ)” as SEQ ID NO: 485.

FIG. 3 shows a set of structures for particular embodiments of linkers which can be linked to amine groups of therapeutic drugs.

FIG. 4 shows a structure of one embodiment of a PSMA-activated thapsigargin prodrug. FIG. 4 discloses SEQ ID NO: 487.

FIG. 5 depicts the complete map of FAP cleavage sites within an 8.5 kDa fragment of recombinant human gelatin prepared from human collagen I. FIG. 5 discloses SEQ ID NOS 221, 237, 238, 222, 239, 223, 240, 224, 241, 242, 225, 243, 226, 244, 227, 245, 228, 246, 229, 247, 230, 248, 231, 249, 232, 250, 233, 251, 234, 252, 235, 253, 236, and 254, respectively, in order of appearance.

FIG. 6 depicts the complete map of FAP cleavage sites within 100 kDa recombinant human gelatin prepared from human collagen I. FIG. 6 discloses the left column Cleavage Fragments as SEQ ID NOS 358, 384, 359, 385, 360, 386, 361, 387, 362, 388, 363, 389, 364, 390, 365, 391, 366, 392, 367, 393, 368, 394, 369, 395, 370, 396, 371, 397, 372, 398, 373, 399, 374, 400, 375, 401, 376, 402, 377, 403, 378, 404, 379, 405, 380, 406, 381, 407, 382, 408, 383, and 409, respectively, in order of appearance. FIG. 6 discloses the right column Cleavage Fragments as SEQ ID NOS 410, 435, 411, 436, 412, 437, 413, 438, 414, 439, 415, 440, 416, 441, 417, 442, 418, 443, 419, 444, 420, 445, 421, 446, 422, 447, 423, 448, 424, 449, 425, 450, 426, 451, 427, 452, 428, 453, 429, 454, 430, 455, 431, 456, 432, 457, 433, 458, 434, and 459, respectively, in order of appearance.

FIG. 7 shows a chemical structure of thapsigargin analog modified in O-8 position with 12-aminododecanoyl side chain coupled to carboxyl-group of an amino acid.

FIG. 8 is a schematic drawing showing sequential PSMA hydrolysis of a 12ADT-Asp-Glu*Glu*Glu*Glu (SEQ ID NO: 486) prodrug (G-202).

FIG. 9 shows Table 5, which provides pharmacokinetic parameters for one embodiment of the injectable emulsion compositions of the invention.

FIG. 10 shows concentrations versus time compared by animal for one embodiment of the injectable emulsion compositions of the invention.

FIG. 11 shows the mean concentration verses time for one embodiment of the injectable emulsion compositions of the invention.

### DETAILED DESCRIPTION OF THE INVENTION

The present invention is a therapeutic prodrug composition in an injectable emulsion that delivers a therapeutic drug directly to a target cell within tumors or at the tumor site. The therapeutic drug may have non-specific toxicity to cells, but is linked to peptides that are cleaved by tissue-specific proteases, such as FAP, PSMA, PSA and hK2. In one embodiment, the therapeutic prodrugs of the present invention are formulated in a safe and commercially feasible injectable emulsion composition that is (a) free of any water-soluble and toxic solubilizer, such as organic solvents or surfactants, (b) sufficiently stable to provide an acceptable shelf life, (c) composed of small oil droplets, (d) filterable though a 0.2-micron filter, (e) transparent or translucent, and (f) lyophilizable.

Following long-standing patent law convention, the terms “a,” “an,” and “the” refer to “one or more when used in this application, including the claims. Thus, for example, reference to “a subject” includes a plurality of subjects, unless the context clearly is to the contrary (e.g., a plurality of subjects), and so forth.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +/−100%, in some embodiments +/−50%, in some embodiments +/−20%, in some embodiments +/−10%, in some embodiments +/−5%, in some embodiments +/−1%, in some embodiments +/−0.5%, and in some embodiments +/−0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

As used herein the term “fibroblast-activation protein-alpha” (FAP) refers to fibroblast-activation protein-alpha as well as other proteases that have the same or substantially the same proteolytic cleavage specificity as FAP. As used herein, the term “side chain” refers to the side chains of amino acids known in the art as occurring in proteins, including those produced by post-translational modifications of amino acid side chains.

As used herein, the term “prostate specific membrane antigen” (PSMA) means prostate specific membrane antigen, as well as all other proteases that have the same or substantially the same proteolytic cleavage specificity as prostate specific membrane antigen.

As used herein the term “human glandular kallikrein 2” (hK2) means human glandular kallikrein 2, as well as other proteases that have the same or substantially the same proteolytic cleavage specificity as hK2.

As used herein, the term “prostate specific antigen” (PSA) means prostate specific antigen, as well as all other proteases that have the same or substantially the same proteolytic cleavage specificity as prostate specific antigen.

As used herein, “sufficiently toxic” refers to therapeutic drugs which display nonspecific toxicity toward cells with an LC₅₀ concentration (that is, the concentration required to kill 50% of clonogenic cells) that is at least 3 times lower than the LC₅₀ concentration of the prodrugs of the invention, more preferably at least 20 times lower, and therapeutic drugs most preferably have an LC₅₀ concentration that is at least 100 times lower than the LC₅₀ concentration of the prodrugs of the invention.

The term “contacting” refers to exposing tissue to the peptides, therapeutic drugs or prodrugs of the invention so that they can effectively inhibit cellular processes, or kill cells.

By “peptide” or “polypeptide” is meant any chain of amino acids, regardless of length or post-translational modification (e.g., glycosylation or phosphorylation). As written herein, amino acid sequences are presented according to the standard convention, namely that the amino-terminus of the peptide is on the left, and the carboxy terminus on the right. “Amino acid sequence” and terms, such as “polypeptide” or “protein,” are not meant to limit the amino acid sequence to the complete, native amino acid sequence associated with the recited protein molecule.

The term “acidifying agent” as used herein refers to acidic agent such as hydrochloric acid or sulfuric acid which is used to adjust pH downward of an emulsion to enhance stability of the emulsion.

The term “alkalizing agent” as used herein refers to basic agent such as sodium hydroxide or preferably arginine which is used to adjust pH upward of an emulsion to enhance stability of the emulsion.

The term “antioxidant” as used in this invention refers to primarily metal ion chelators that are safe to use in an injectable product. A metal ion chelator works as an antioxidant by binding to metal ions and thereby reduces the catalytic effect of metal ions on the oxidation reaction of the test substance. Metal chelators that are useful in this invention may include EDTA, glycine and citric acid or salts thereof.

A “preservative” as used in this invention refers to any preservative selected from the group comprising a cresol, paraben, phenol, benzalkonium chloride, benzoic acid, benzoate, benzyl alcohol, chlorobutanol, thimerosal, sorbic acid, sorbate, EDTA or a combination thereof, or any similar compounds that would be recognized by one of ordinary skill in the art.

A composition is “chemically stable” if the active compound in the composition is not substantially chemically degraded after storage under appropriate conditions for at least one month. In certain embodiments, the concentration of the intact active compound in the composition is reduced by less than about 10%, preferably less than about 8%, preferably less than about 6%, and more preferably less than about 5% under appropriate storage conditions (e.g., at −20° C., 2-8° C., or at room temperature) for at least 3 months.

The term “cryo-protectant” as used in this invention refers to any of the safe and biocompatible agent(s) that protect the emulsion during freezing by keeping the sub-micron size droplets separate in the surrounding milieu. The cryo-protectants useful for this invention include, but are not limited to, monosaccharides, disaccharides, polysaccharides, poly-ols, or mixtures thereof, or any other similar compound that would be recognized by one of ordinary skill in the art. For instance, in certain embodiments, the cryo-protectant sucrose, trehalose, maltose, or a mixture thereof. In certain embodiments, the cryo-protectant is sucrose, a combination of sucrose and mannitol, or a combination of sucrose and trehalose.

The term “emulsion” as used herein refers to an oil-in-water emulsion.

The term “injectable” as used in this invention refers to the acceptance of an ingredient by a drug regulating authority (e.g., the U.S. Food and Drug Administration) permitting its use in an injection drug.

The term “lecithin” as used herein is a naturally occurring mixture of the diglycerides of stearic, palmitic, and oleic acids, linked to the choline ester of phosphoric acid, commonly called phosphatidylcholine. According to the United States Pharmacopoeia (USP), lecithin is a non-proprietary name describing a complex mixture of acetone-insoluble phospholipids, which consists primarily of phosphatidylcholine, phosphatidylethanolamine, phosphatidylserine and phosphatidylinositol, combined with various amounts of other substances such as triglycerides, fatty acids, and carbohydrates. The lecithin useful in the present invention includes soy lecithin, egg lecithin, hydrogenated soy lecithin, hydrogenated egg lecithin, and combinations thereof. Lecithin is used as an emulsifier in the emulsion of the present invention.

The term “light transmittance (%)” as used herein is a measurement of transparency of the emulsion and is defined as the fraction of incident light at a specified wavelength (e.g., 600 nm) that passes through a sample. It is calculated using the following equation:

T_{λ}=I÷I₀×100

where I₀ is the intensity of the incident light and I is the intensity of the light coming out of the sample and T_{λ} is transmittance. The T_{λ} value can be readily measured by a UV-visible spectrophotomer at a fixed wavelength. A visible wavelength such as 600 nm is commonly used.

The light transmittance value of an emulsion is directly related to its droplet size and is one aspect of the present invention that can be used to differentiate the emulsion of this invention from an emulsion of the prior art. For a prior art emulsion such as Propofol Injectable Emulsion, the light transmittance value measured at 600 nm wavelength is generally less than 5-10%, which is due to the light-reflecting white and opaque properties of these emulsions.

The term “medium chain triglyceride” (MCT) as used herein refers to another class of triglyceride oil that can be either naturally derived or synthetically produced. MCTs are made from fatty acids that are usually about 8 to about 12 carbons in length. Like vegetable oils, MCTs have been used extensively in injectable emulsion preparations as a source of calories for patients requiring parenteral nutrition. Such oil is commercially available as Miglyol 812 from SASOL GmbH, Germany, CRODAMOL GTCC-PN from Croda, Inc. of Parsippany, N.J., or Neobees M-5 oil from PVO International, Inc., of Boonton, N.J. Other low-melting medium chain oils may also be used in the present invention.

As used herein, an emulsion composition of the invention is “physically stable” if it can be stored as a lyophilized emulsion under appropriate conditions for at least 3 months and, upon reconstitution, does not demonstrate an increase in its average droplet size by more than about 100%, or is without evidence of phase separation, creaming, or aggregation. In certain embodiments, the average size of droplets of a composition of the present invention does not increase by more than about 100%, preferably more than about 75%, preferably more than about 50%, preferably more than about 40%, preferably more than about 30%, preferably more than about 25%, preferably more than about 20%, or preferably more than about 10% under appropriate storage conditions (e.g., at −20° C., 2-8° C., or room temperature) for 3 months.

As used herein, “vegetable oils” include, but are not limited to, almond oil, borage oil, black currant seed oil, corn oil, safflower oil, sesame oil, cottonseed oil, peanut oil, olive oil, rapeseed oil, coconut oil, palm oil, canola oil, etc. may be used as well. The specific type of vegetable oil used (e.g., soybean oil, corn oil, or safflower oil, etc.) is not critical, so long as it is safe, well tolerated, pharmaceutically acceptable, chemically stable and can be formed into droplets having a desired size range.

As an example, the term “soybean oil” as used herein refers to refined oil extracted from soybean. As another example, “almond oil” as used herein refers to refined oil extracted from almond, and so forth. For injection use, all such oils used in the present invention must pass certain quality specifications including purity, microbiological and endotoxin limits, meeting certain compendial standards and be manufactured in a facility meeting cGMP standards.

In certain embodiments, the vegetable oil to MCT oil ratio is within a range of about 2:1 to about 1:2, preferably about 1:1.

The term “osmotic pressure modifying agent” as used herein refers to sucrose, glycerol, or a mixture thereof. In certain embodiments, the emulsion of the present invention has an osmotic pressure or osmolality of approximately 300 to 1000 mOsm.

The term “antibody” refers to intact immunoglobulin molecules as well as to fragments thereof, such as Fab, F(ab′)₂, and Fv fragments, which are capable of binding an epitopic determinant. Antibodies that bind to the polypeptides of the present invention, for example, FAP, PSMA, PSA or hK2, can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “biologically active” refers to a protein having structural, regulatory, or biochemical functions of a naturally occurring molecule. Likewise, “immunologically active” or “immunogenic” refers to the capability of a natural, recombinant, or synthetic protease (for example, FAP, hK2, PSMA, or PSA), or of any oligopeptide thereof, to induce a specific immune response in appropriate animals or cells and to bind with specific antibodies.

“Conservative amino acid substitutions” and “conservative variations” are those substitutions and variations that are predicted to least interfere with the properties of the original protein, e.g., the structure and especially the function of the protein is conserved and not significantly changed by such substitutions. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “detectable label” refers to a reporter molecule or enzyme that is capable of generating a measurable signal and is covalently or noncovalently joined to a polynucleotide or polypeptide.

The term “substantially purified” refers to nucleic acid or amino acid sequences that are removed from their natural environment and are isolated or separated, and are at least about 60% free, preferably at least about 75% free, preferably at least about 80% free, and most preferably at least about 90% free from other components with which they are naturally associated.

The terms “treat” or “treatment” refer to both therapeutic and prophylactic or preventative measures, wherein the object is to prevent or slow down (lessen) an undesired physiological change or disorder, such as the development or spread of a proliferative disorder, including cancer. For purposes of this invention, beneficial or desired clinical results include, but are not limited to, alleviation of symptoms, diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have or having the condition or disorder or those in which the condition or disorder is to be prevented. The terms “treating”, “treat”, or “treatment” embrace both preventative, e.g., prophylactic, and palliative treatment.

The phrases “therapeutically effective amount” and “therapeutically effective dose” mean an amount of an active compound of the present invention that (i) treats or prevents the particular disease, condition, or disorder, (ii) attenuates, ameliorates, or eliminates one or more symptoms of the particular disease, condition, or disorder, or (iii) prevents, reduces or delays the onset of one or more symptoms of the particular disease, condition, or disorder described herein. The reduction need not be complete. That is, a partial reduction in the symptom is contemplated. Additionally, the symptom need not be reduced permanently. A temporary reduction in at least one symptom is contemplated by the present invention. In the case of cancer, the therapeutically effective amount of the drug may reduce the number of cancer cells or tumor-associated cells; reduce the tumor size; inhibit (e.g., slow to some extent and preferably stop) cancer cell infiltration into peripheral organs; inhibit (e.g., slow to some extent and preferably stop) tumor metastasis; inhibit, to some extent, tumor growth; show improvement in biological markers associated with the cancer; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, melanoma, lymphoma, blastoma, sarcoma, and leukemia or lymphoid malignancies. More particular examples of such cancers include, but are not limited to, epithelial cancers, cancer of the breast, cervix, liver, ovary, prostate, lung, colon and rectum, pancreas, stomach or kidney. Additional examples of cancer are provided in U.S. application Ser. No. 12/087,398 and generally known in the art.

The term “cell proliferative disorder” denotes malignant (i.e., cancerous) as well as non-malignant cell populations which often appear to differ from the surrounding tissue both morphologically and genotypically, for example, as in hyperplasia (such as benign prostatic hyperplasia (BPH)), and malignant and benign neoplasms. Malignant cells (i.e., cancer) may develop (but their development is not required for the invention described herein) from these cell populations as a result of a multistep process. Thus, as used herein, “cell proliferative disorder” includes, but is not limited to, cancer.

The term “cell associated with a tumor” or “tumor-associated cell” as used herein means non-transformed cells that become part of the tumor mass as opposed to the cancer cells themselves, for example, tumor endothelial cells, stromal cells, fibroblasts or cells of the vascular endothelium.

The term “prodrug” as used in this application refers to a precursor or derivative form of a pharmaceutically active substance that is less cytotoxic to tumor cells compared to the parent drug and is capable of being enzymatically or hydrolytically activated or converted into the more active parent form. See, e.g., Wilman, “Prodrugs in Cancer Chemotherapy”, Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but arc not limited to, phosphate-containing prodrugs, thiophosphatc-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described herein.

The term “protecting group” or “Pg” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include acetyl, trifluoroacetyl, t-butoxycarbonyl (Boc), benzyloxycarbonyl (CBz) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see T. W. Greene, Protective Groups in Organic Synthesis, John Wiley & Sons, New York, 1991.

The term “animal” refers to humans (male or female), non-human primates, companion animals (e.g., dogs, cats and horses), food-source animals (e.g., cows, pigs, sheep and poultry), zoo animals, marine animals, birds, rodents and other similar animal species.

The term “pharmaceutically acceptable” with respect to a component, such as a salt, carrier, excipient or diluent of a composition according to the presently disclosed subject matter refers to a component that is compatible with the other ingredients of the composition in that it can be combined with the presently disclosed active compounds without eliminating the therapeutic efficacy of the compounds and is suitable for use with subjects as provided herein without undue adverse side effects (including, but not limited to, toxicity, irritation, and allergic response) to the subject to which the particular compound is administered. Examples of pharmaceutically acceptable components include, without limitation, any of the standard pharmaceutical carriers, such as phosphate buffered saline solutions, water, emulsions, such as oil/water emulsion, microemulsions, and various types of wetting agents.

The term “non-naturally occurring amino acid” refers to amino acids that are not normally found in living organisms.

The term “at least one symptom is reduced” means that, after treatment at least one of any number of symptoms is reduced. The reduction need not be complete. That is, a partial reduction in the symptom is contemplated. Additionally, the symptom need not be reduced permanently. A temporary reduction in at least one symptom is contemplated by the present invention.

The term “subject” refers to any animal which is to be the recipient of a particular treatment, or from whom cancer stem cells are harvested. Typically, the terms “subject” and “patient” are used interchangeably, unless indicated otherwise herein.

As used herein, the term “subject suspected of having cancer” refers to a subject that presents one or more signs or symptoms indicative of a cell proliferative disorder or cancer (e.g., a noticeable lump or mass) or is being screened for a cell proliferative disorder or cancer (e.g., during a routine physical). A subject suspected of having cancer may also have one or more risk factors for a cell proliferative disorder or cancer. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention. A “preliminary diagnosis” is one based only on visual (e.g., CT scan or the presence of a lump) and antigen tests.

The term “subject at risk for cancer” is a person or patient having an increased chance of a cell proliferative disorder or cancer (relative to the general population). Such subjects may, for example, be from families with a history of a cell proliferative disorder or cancer. In another example, subjects at risk may be individuals in which there is a genetic history of a particular cancer associated with race, nationality or heritage or exposure to an environmental trigger.

As used herein, the term “administration” refers to the act of giving a drug, prodrug, active compound, or other agent, or therapeutic treatment (e.g., compositions of the present invention) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

As used herein, the term “co-administration” refers to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

Protease-Specific Peptides

Novel classes of peptides that contain a cleavage site specific for, for example, FAP, hK2, PSMA and PSA have been described. See e.g., U.S. Pat. Nos. 7,906,477; 7,053,042; 8,450,280; 7,767,648; 7,468,354; 6,265,540; 6,504,014; 6,410,514; 6,545,131; and 7,635,682; and U.S. patent application Ser. Nos. 12/087,398 and 13/471,316. These peptides, and other peptides that have cleavage sites specific for a selected protease, are useful for substantially inhibiting the non-specific toxicity of therapeutic agents prior to the agents contacting a tissue containing the selected protease. In some embodiments, such tissues exhibit signs of a cell proliferative disorder, and may be cancerous.

In one aspect the invention features a peptide containing an amino acid sequence that includes a cleavage site specific for a selected protease, for example, PSMA, PSA, hK2 or FAP, or an enzyme having a proteolytic activity of the selected protease. The peptides of the invention are preferably not more than 20 amino acids in length, more preferably not more than about 10 amino acids in length, and even more preferably not more than about 6 amino acids in length. In one embodiment, the preferred amino acid sequences of the invention are linear. In one embodiment of the invention the amino acid sequence may be cyclical such that the cyclical form of the sequence is an inactive drug that can become an activated drug upon cleavage by a selected protease and linearization.

Although the invention encompasses any peptide prodrug that may be selectively cleaved by a protease associated with a selected proteolytic activity, the following examples for FAP, PSMA, PSA and hK2 are illustrative.

PSA Peptides

The invention features prodrugs that include a peptide containing an amino acid sequence that includes a cleavage site specific for PSA or an enzyme having a proteolytic activity of PSA. Cleavage sites for PSA have been described, for example, in U.S. Pat. Nos. 6,265,540; 6,410,514; 6,504,014; 6,545,131 and 7,635,682. For example, the cleavage site recognized by PSA is flanked by at least an amino acid sequence, X₅X₄X₃X₂X₁. This peptide contains the amino acid glutamine, asparagine or tyrosine at position X₁. X₂ can be leucine, tyrosine, or lysine. X₃ can be serine or lysine. X₄ can be serine, isoleucine, or lysine. X₅ can be from 0 to 16 further amino acids. Some preferred embodiments include a sequence for X₅ that is substantially identical to the 16 remaining amino acids in the wild type semenogelin I or semenogelin II sequence. The amino acid sequence can further comprise X₋₁, which is linked to the carboxy terminus of X₁ to create the amino acid sequence X₅X₄X₃X₂X₁X₋₁. X₋₁ is up to 10 further amino acids. Preferably, X₋₁ has histidine, leucine threonine or serine linked to the carboxy terminus of X₁. The PSA cleavage site is located at the carboxy terminal side of X₁, unless X₋₁ has histidine linked to the carboxy terminus of X₁, in which case the PSA cleavage site is to the carboxy terminal side of histidine.

Another amino acid sequence is X₆X₅X₄X₃X₂X₁ in which X₅ is serine or lysine, X₆ is from 0 to 15 further amino acids, and the other amino acids are as above. X₋₁ can also be present, as noted above. Another amino acid sequence is X₇X₆X₅X₄X₃X₂X₁, in which X₆ is histidine or asparagine X₇ is from 0 to 14 further amino acids, and the other amino acids are as above. X₁ can also be present, as noted above.

Some examples of preferred peptides include tetraamino acid sequences such as Ser-Lys-Leu-Gln (SEQ ID NO: 1), Ile-Ser-Tyr-Gln (SEQ ID NO: 2), and Lys-Ser-Lys-Gln (SEQ ID NO: 3). Some examples of preferred pentaamino acid sequences are Ser-Ser-Lys-Leu-Gln (SEQ ID NO: 4), Lys-Ile-Ser-Tyr-Gln (SEQ ID NO: 5), and Thr-Lys-Ser-Lys-Gln (SEQ ID NO: 6). Some examples of preferred hexaamino acid sequences are His-Ser-Ser-Lys-Leu-Gln (SEQ ID NO: 7), Asn-Lys-Ile-Ser-Tyr-Gln (SEQ ID NO: 8), and Ala-Thr-Lys-Ser-Lys-Gln (SEQ ID NO: 9). Some examples of preferred heptaamino acid sequences are Glu-His-Ser-Ser-Lys-Leu-Gln (SEQ ID NO: 10), Gln-Asn-Lys-Ile-Ser-Tyr-Gln (SEQ ID NO: 11), Glu-Asn-Lys-Ile-Ser-Tyr-Gln (SEQ ID NO: 12), Ala-Thr-Lys-Ser-Lys-Gln-His (SEQ ID NO: 13), and His-Ser-Ser-Lys-Leu-Gln-Leu (SEQ ID NO: 481). As noted, further amino acids can comprise X₋₁.

Other examples of peptides that contain a cleavage site specific for PSA include peptides consisting of or comprising the amino acid sequence Ser-Ser-Lys-Tyr-Gln (SSKYQ) (SEQ ID NO: 18), Gly-Lys-Ser-Gln-Tyr-Gln (GKSQYQ) (SEQ ID NO: 19) and Gly-Ser-Ala-Lys-Tyr-Gln (GSAKYQ) (SEQ ID NO: 20).

In one embodiment, the thapsigargin derivative 8-O-(12-[L-leucinoylamino]dodecanoyl)-8-O-debutanoylthapsigargin (L I 2ADT) is linked to the carboxy terminus of a PSA-specific amino acid sequence.

hK2 Peptides

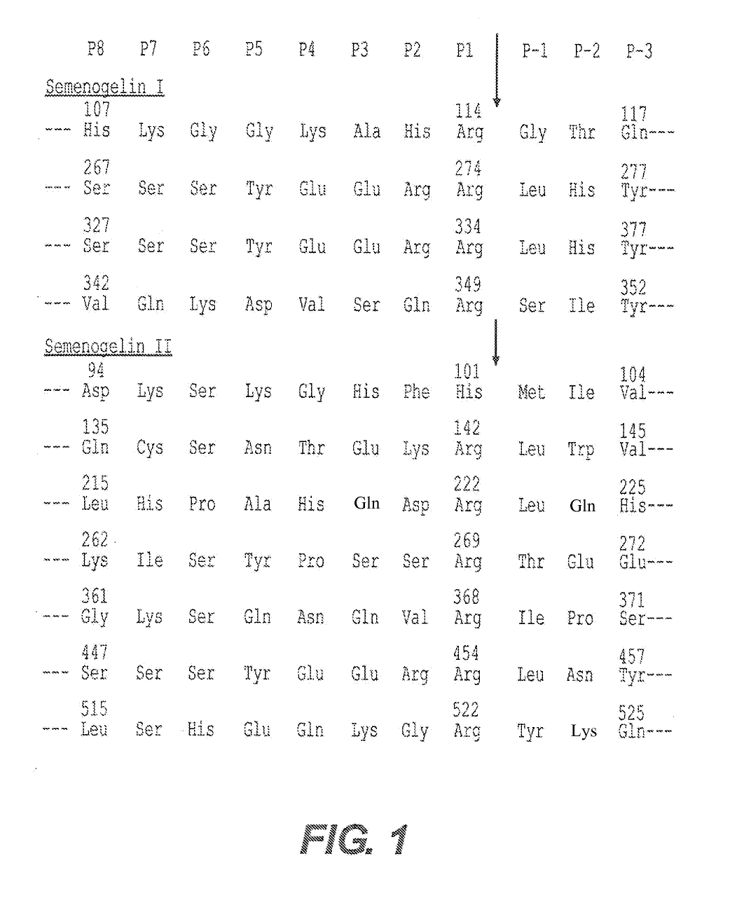

The invention also features prodrugs that include a peptide containing an amino acid sequence that includes a cleavage site specific for hK2 or an enzyme having a proteolytic activity of hK2. Cleavage sites for hK2 have been described, for example, in U.S. Pat. Nos. 7,906,477; 7,053,042 and 8,450,280. FIG. 1, for example, shows a portion of the amino acid sequence of Semenogelin I (SEQ ID NOs: 21-24) and Semenogelin II (SEQ ID NOs: 25-31), showing the cleavage sites for human kallikrein 2.

The cleavage site recognized by hK2 is flanked by at least an amino acid sequence, X₄X₃X₂X₁. This oligopeptide contains the amino acid arginine, histidine or lysine at position X₁. X₂ can be arginine, phenylalanine, lysine, or histidine. X₃ can be lysine, serine, alanine, histidine or glutamine. X₄ can be from 0 to 20 further amino acids, preferably at least two further amino acids. Some preferred embodiments include a sequence for X₄ that is substantially identical to the 20 amino acids in the wild type semenogelin I or semenogelin II sequence that are the from fourth to twenty fourth amino acids to the N-terminal side of recognized semenogelin cleavage sites. The amino acid sequence can further comprise X₋₁, which is linked to the carboxy terminus of X₁ to create the amino acid sequence X₄X₃X₂X₁X₋₁. X₋₁ is up to a further 10 amino acids, and can include any amino acids. Preferably X₁ has leucine, alanine or serine linked to the carboxy terminus of X₁. X₋₁ can include L- or D-amino acids. The hK2 cleavage site is located at the carboxy terminal side of X₁.

In some preferred peptides, both X₁ and X₂ are arginine

Some examples of preferred peptides include (Note that the symbol][denotes an hK2 cleavage site):

```
  1. Lys-Arg-Arg][(SEQ ID NO: 32)
  2. Ser-Arg-Arg][(SEQ ID NO: 33)
  3. Ala-Arg-Arg][(SEQ ID NO: 34)
  4. His-Arg-Arg][(SEQ ID NO: 35)
  5. Gln-Arg-Arg][(SEQ ID NO: 36)
  6. Ala-Phe-Arg][(SEQ ID NO: 37)
  7. Ala-Gln-Arg][(SEQ ID NO: 38)
  8. Ala-Lys-Arg][(SEQ ID NO: 39)
  9. Ala-Arg-Lys][(SEQ ID NO: 40)
10. Ala-His-Arg][(SEQ ID NO: 41)
```

Additional preferred peptides of longer sequence length include:

11. Gln-Lys-Arg-Arg][(SEQ ID NO: 42)
12. Lys-Ser-Arg-Arg][(SEQ ID NO: 43)
13. Ala-Lys-Arg-Arg][(SEQ ID NO: 44)
14. Lys-Lys-Arg-Arg][(SEQ ID NO: 45)
15. His-Lys-Arg-Arg][(SEQ ID NO: 46)
16. Lys-Ala-Phe-Arg][(SEQ ID NO: 47)
17. Lys-Ala-Gln-Arg][(SEQ ID NO: 48)
18. Lys-Ala-Lys-Arg][(SEQ ID NO: 49)
19. Lys-Ala-Arg-Lys][(SEQ ID NO: 50)
20. Lys-Ala-His-Arg][(SEQ ID NO: 51)

Additional preferred peptides that include an X₋₁ amino acid are:

21. Lys-Arg-Arg][Leu (SEQ ID NO: 52)
22. Ser-Arg-Arg][Leu (SEQ ID NO: 53)
23. Ala-Arg-Arg][Leu (SEQ ID NO: 54)
24. Ala-Arg-Arg][Ser (SEQ ID NO: 55)
25. His-Arg-Arg][Ala (SEQ ID NO: 56)
26. Gln-Arg-Arg][Leu (SEQ ID NO: 57)
27. Ala-Phe-Arg][Leu (SEQ ID NO: 58)
28. Ala-Gln-Arg][Leu (SEQ ID NO: 59)
29. Ala-Lys-Arg][Leu (SEQ ID NO: 60)
30. Ala-Arg-Lys][Leu (SEQ ID NO: 61)
31. Ala-His-Arg][Leu (SEQ ID NO: 62)

Preferred peptides of still longer sequence length having X₋₁ include:

32. His-Ala-Gln-Lys-Arg-Arg][Lcu (SEQ ID NO: 63)
33. Gly-Gly-Lys-Ser-Arg-Arg][Leu (SEQ ID NO: 64)
34. His-Glu-Gln-Lys-Arg-Arg][Leu (SEQ ID NO: 65)
35. His-Glu-Ala-Lys-Arg-Arg][Leu (SEQ ID NO: 66)
36. Gly-Gly-Gln-Lys-Arg-Arg][Leu (SEQ ID NO: 67)
37. His-Glu-Gln-Lys-Arg-Arg][Ala (SEQ ID NO: 68)
38. Gly-Gly-Ala-Lys-Arg-Arg][Leu (SEQ ID NO: 69)
39. His-Glu-Gln-Lys-Arg-Arg][Scr (SEQ ID NO: 70)
40. Gly-Gly-Lys-Lys-Arg-Arg][Leu (SEQ ID NO: 71)
41. Gly-Gly-His-Lys-Arg-Arg][Leu (SEQ ID NO: 72)

Other embodiments of peptide sequences which are useful for cleavage by hK2 and proteases with the hydrolytic activity of hK2 are disclosed in the Sequence Listing (SEQ ID NOs: 73-142)

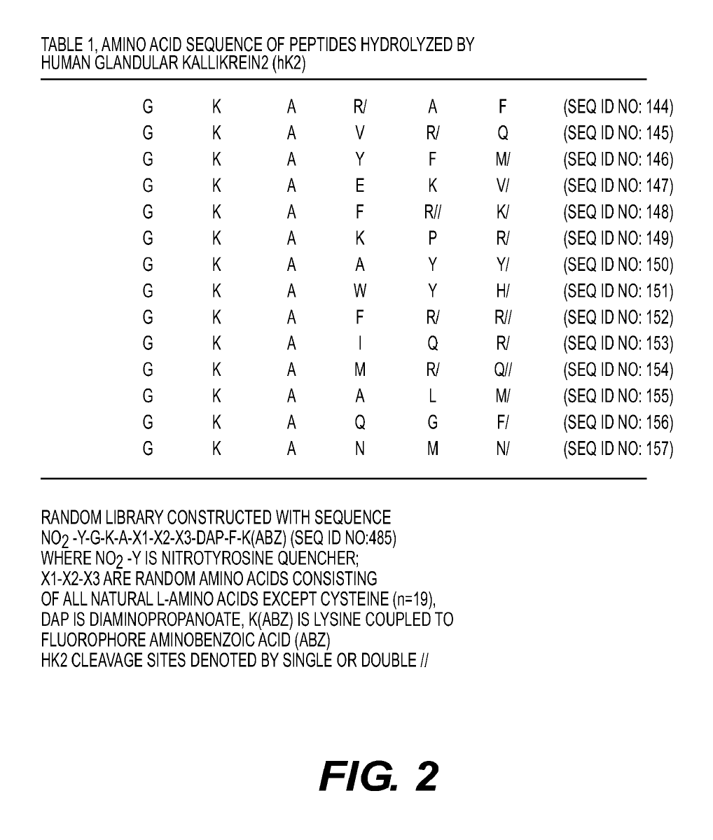

In another preferred embodiment, the hK2 peptide sequences of the invention comprise the sequence G-K-A-X₁-X₂-X₃ (SEQ ID NO: 143), wherein at least one of X₁, X₂, and X₃ is an arginine residue and wherein the amino acid residues at the other two positions of X₁, X₂, and X₃ arc any amino acid residue. hK2 may cleave the peptide after either X₁, X₂, or X₃, and in one preferred embodiment, hK2 cleaves the peptide after an arginine residue. Specific preferred sequences (including cleavage sites) are shown in FIG. 2 (SEQ ID NOS: 144-157). Further preferred sequences are included in the sequences shown in FIG. 2, with an additional leucine residue after the X₃ position (SEQ ID NOS: 158-171).

PSMA Peptides

The invention also features prodrugs that include a peptide containing an amino acid sequence that includes a cleavage site specific for PSMA or an enzyme having a proteolytic activity of PSMA. Cleavage sites for PSMA have been described, for example, in U.S. Pat. Nos. 7,767,648 and 7,468,354. Treatment methods using a PSMA activated prodrug have been described in PCT/US13/56523. Further, methods for making PSMA activated prodrugs are disclosed in U.S. Application No. 61/791,909. Prodrugs are designed that can be activated by the pteroyl poly-γ-glutamyl carboxypeptidase (folate hydrolase) activity of PSMA. Gamma glutamyl hydrolase (GGH) is secreted by hepatocytes and by a variety of tumor cell types and GGH activity is present in human serum. Therefore, effective side chain-linked substrates are desirably specifically hydrolyzed by PSMA with minimal hydrolysis by GGH.

The PSMA cleavage site includes at least the dipeptide, X₁X₂. This peptide comprises the amino acids Glu, Asp, Gln or Asn at position X₁, however in a preferred embodiment X₁ comprises the amino acids Glu or Asp. X₂ can be Glu, Asp, Gln, or Asn. The amino acid sequence can further comprise X₃, which is linked to the carboxy terminus of X₂. X₃ may be Glu, Asp, Gln or Asn. The amino acid sequence can further comprise X₄, which is linked to the carboxy terminus of X₃. X₄ may be Glu, Asp, Gln or Asn. The amino acid sequence can further comprise X₅, which is linked to the carboxy terminus of X₄. X₅ may be Glu, Asp, Gln or Asn. The amino acid sequence may further comprise X₆, which is linked to the carboxy terminus of X₅. X₆ may be Glu, Asp, Gln or Asn. Further peptides of longer sequence length can be constructed in similar fashion.

For example, one embodiment of the present invention includes the peptide sequence X₁X₂X₃X₄X₅X₆X₇, wherein X₁, X₂, X₃, X₄, X₅ and X₆ arc as defined above, and X₇ is up to 24 further amino acids, preferably up to 14 amino acids, and more preferably up to 9 further amino acids.

In another example, the PSMA peptides of the present invention are of the following sequence: X₁ . . . Xₙ, where n is any integer ranging from 2 to 30, preferably ranging from 2 to 20, more preferably ranging from 2 to 15, and even more preferably ranging from 2 to 6, where X₁ is Glu, Asp, Gln or Asn, but is preferably Glu or Asp, and the remaining peptides up to Xₙ (for example, X₂ where n=2; X₂X₃ where n=3; X₂X₃X₄ where n=4, and so on) are independently selected from Glu, Asp, Gln and Asn. Some preferred examples of peptide sequences are as above. In another example, X₂ to Xₙ₋₁ are independently selected from Glu, and Asp, and Xₙ is independently selected from Glu, Asp, Gln and Asn.

The length of the PSMA peptides of the present invention can be optimized to allow for efficient PSMA hydrolysis, enhanced solubility of therapeutic drug in aqueous solution, if this is needed, and limited non-specific cytotoxicity in vitro.

Among the α-linked dipeptides, Asp-Glu, Asp-Asp, Asp-Asn and Asp-Gln are preferably employed for use in the PSMA prodrugs described herein. Among the all α-linked tripeptides, Glu-Glu-Glu, Glu-Asp-Glu, Asp-Glu-Glu, Glu-Glu-Asp, Glu-Asp-Asp, Asp-Glu-Asp, Asp-Asp-Glu, Asp-Asp-Asp, Glu-Glu-Gln, Glu-Asp-Gln, Asp-Glu-Gln, Glu-Glu-Asn, Glu-Asp-Asn, Asp-Glu-Asn, Asp-Asp-Gln, and Asp-Asp-Asn are preferably employed for use in the PSMA prodrugs described herein. Tripeptides containing Gln or Asn in positions X₂ can also be desirably employed. Longer all α-linked peptides may also be employed for use in the prodrugs described herein, and such peptides with Gln or Asn in any positions X₂ to Xₙ can also be desirably employed.

Side-Chain Linkages

PSMA is also able to hydrolyze a variety of side chain-linked peptides. Particular side chain-linked, for example, γ-linked peptides, are not specific for PSMA but can also be hydrolyzed by GGH. Some preferred peptides take advantage of the dual ability of PSMA to hydrolyze certain α- and side-chain linkages between aspartyl, and glutamyl residues.

Among the side chain-linked dipeptides, Glu*Asp, Glu*Asn, Glu*Glu,

Glu*Gln, Asp*Asp, Asp*Glu, Asp*Asn, and Asp*Gln can be employed for use in the PSMA prodrugs described herein. Among the all side chain-linked tripeptides, Glu*Glu*Glu, Glu*Asp*Glu, Asp*Glu*Glu, Glu*Glu*Asp, Glu*Asp*Asp, Asp*Glu*Asp, Asp*Asp*Glu, Asp*Asp*Asp, Glu*Glu*Gln, Glu*Asp*Gln, Asp*Glu*Gln, Glu*Glu*Asn, Glu*Asp*Asn, Asp*Glu*Asn, Asp*Asp*Gln, and Asp*Asp*Asn can be preferably employed for use in the PSMA prodrugs described herein. Longer peptides which of analogous sequences can also be employed for use in the PSMA prodrugs described herein.

Mixed Peptides

Some preferred peptides include a PSMA-hydrolyzable, α-linked dipeptide “cap” that are not substrates for GGH, and are more specific PSMA substrates. Combination α- and side chain-linked PSMA substrates can be highly efficient and specific. For example, Glu*Glu*Glu*Asp-Glu (SEQ ID NO: 487), and Glu*Glu*Glu*Asp-Gln (SEQ ID NO: 488) have high stability in serum. Peptides containing two α-linkages and two γ-linkages, for example, Asp-Glu*Glu*Asp-Glu (SEQ ID NO: 489) can be completely stable to hydrolysis in human and mouse plasma. A number of aspartate- and glutamate-containing linkers are depicted in U.S. Pat. No. 7,767,648. These particular linkers can be bonded to amine groups on therapeutic drugs.

The peptides listed are among those that are preferred: Glu*Glu*Glu*Asp-Glu (SEQ ID NO: 487), Asp-Glu*Glu*Asp-Glu (SEQ ID NO: 489), and Glu-Glu*Glu*Asp-Glu (SEQ ID NO: 490). Numerous other peptides with mixed α- and side chain linkages and otherwise corresponding to the description herein can be readily envisioned and constructed by those of ordinary skill in the art.

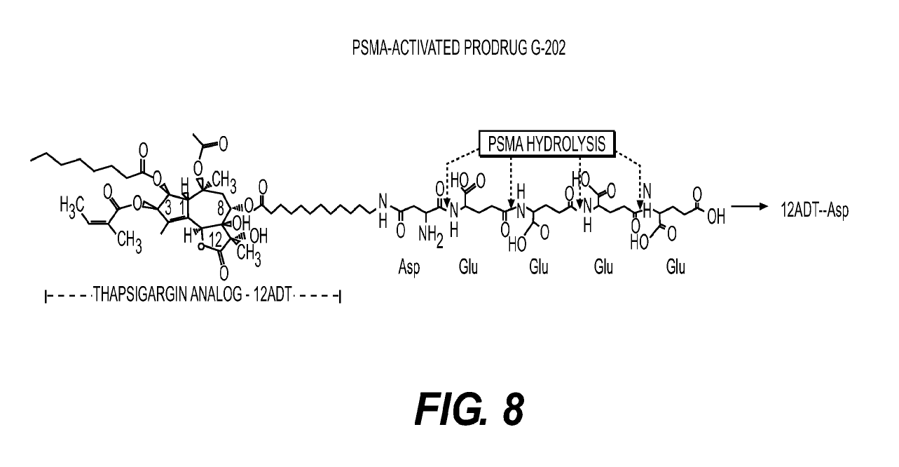

In one embodiment of the present invention, the peptide comprises the sequence Asp-Glu*Glu*Glu*Glu (SEQ ID NO: 486), wherein at least one of the bonds designated with * is a gamma carboxy linkage. In another aspect of the invention, the peptide is linked via the aspartic acid to a composition comprising a therapeutically effective amount of 8-O-(12-aminododecanoyl)-debutanoyl thapsigargin (12ADT), as is shown in FIG. 8. In one embodiment, the prodrug of the present invention is produced by coupling 8-O-(12-aminododecanoyl)-debutanoyl thapsigargin (12ADT) to the beta carboxyl of Asp at the N terminal end of the masking peptide Asp-Glu-γ-Glu-γ-Glu-γ-Glu (wherein the hyphen denotes alpha linkage and gamma symbol denotes gamma linkage) to produce the prodrug 12ADT-β-Asp-α-Glu-γ-Glu-γ-Glu-γ-GluOH. (i.e., G-202) (FIG. 8).

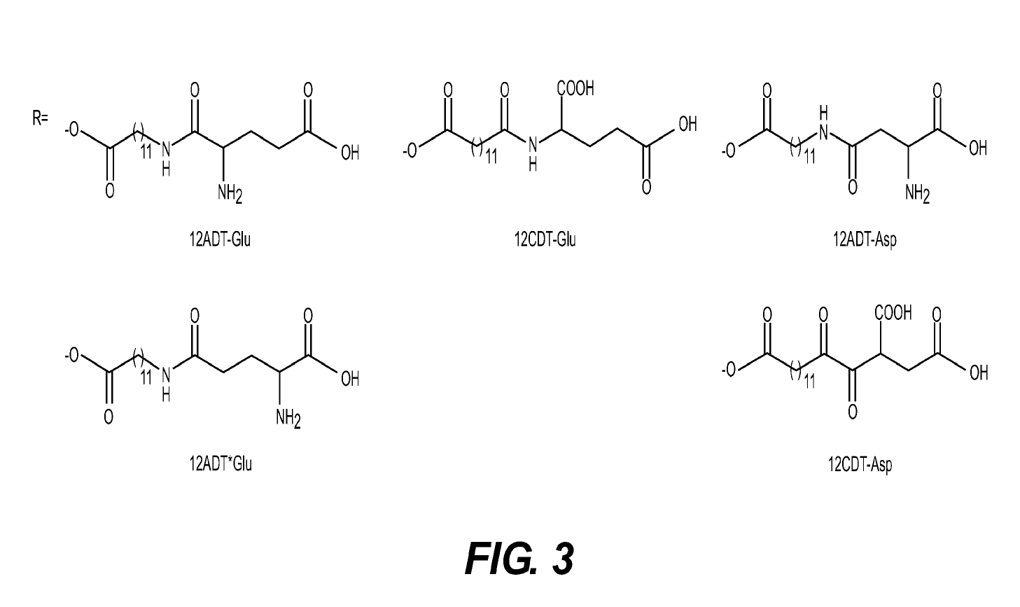

In other embodiments, a dicarboxylic acid linker can be used, such as the 12-carbon linker 12-carboxydodecanoate, shown, in FIG. 3, for example, 12-CDT-Asp.

The peptides of the invention are preferably not more than 20 amino acids in length, preferably not more than ten amino acids in length, more preferably not more than 6 amino acids in length. Some peptides which are only two or three amino acids in length are quite suitable for use in the PSMA prodrugs described herein. Some preferred amino acid sequences of the invention are linear. However, multiple linkage sites present on dicarboxylic amino acids may also be used to produce branched peptides. These branched peptides could include a therapeutic agent coupled to each amino acid of the peptide chain, such that cleavage of individual amino acids from the peptide chain by the enzymatic activity of PSMA releases multiple molecules of therapeutic agent.

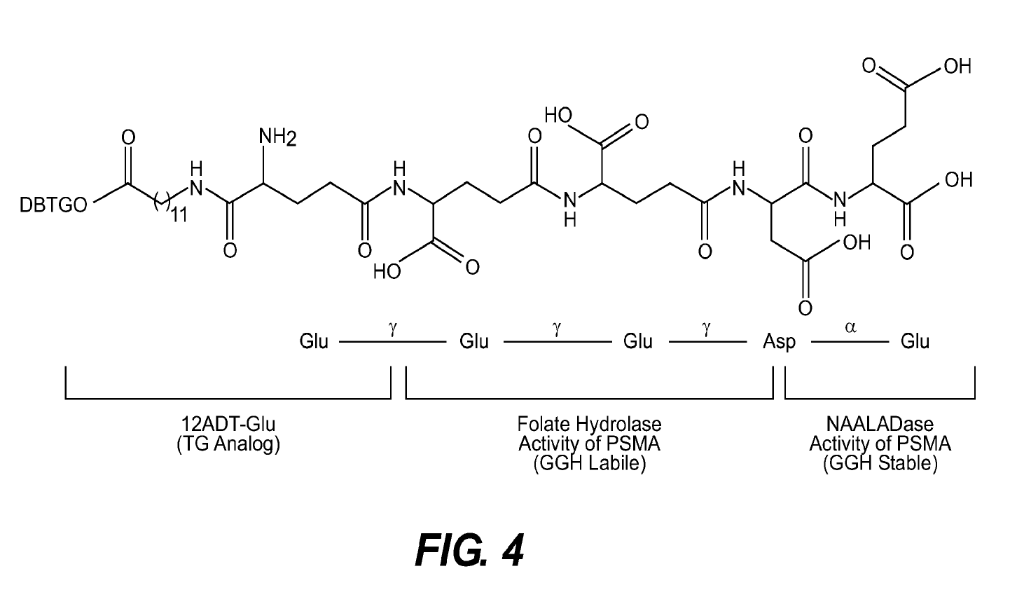

FIG. 4 is a structure of a particular embodiment of a PSMA-activated thapsigargin prodrug. DBTG refers to 8-O-debutanoylthapsigargin, which is linked via the oxygen atom to the remainder of the prodrug as shown. Thus, preferred substrates combine the specificity of the α-linkage with the enhanced efficiency of the T-linkage. The longer-length, negatively-charged, substrates can serve two additional purposes: first, they help to make highly lipophilic toxins, for example, thapsigargin analogs, more water soluble; second, the highly charged prodrug will be less likely to cross the plasma membrane, further limiting non-specific cytotoxicity.

The following prodrugs are examples of preferred embodiments:

(SEQ ID NO: 487)
(1) 12ADT-Glu*Glu*Glu*Asp-Glu
(SEQ ID NO: 490)
(2) 12ADT*Glu-Glu*Glu*Asp-Glu
(SEQ ID NO: 487)
(3) 12CDT-Glu*Glu*Glu*Asp-Glu
(SEQ ID NO: 489)
(4) 12ADT-Asp-Glu*Glu*Asp-Glu
(SEQ ID NO: 489)
(5) 12CDT-Asp-Glu*Glu*Asp-Glu
(SEQ ID NO: 486)
(6) 12ADT-Asp-Glu*Glu*Glu*Glu(FIG. 8)

The prodrugs are hydrolyzed by PSMA and release the corresponding Asp- or Glu-containing thapsigargin analogs, or the thapsigargin analog itself, and also lack potent cytotoxicity when not metabolized by PSMA. Non-PSMA producing TSU-Pr1 human prostate cancer cell line is exposed to each of the PSMA prodrugs at doses that are approximately 50-times the LD₅₀ for the corresponding free thapsigargin analog. Against the TSU prostate cancer cell line, 12ADT-Glu has an LD₅₀ value for killing of about 50 nM.

FAP Peptides

The invention also features prodrugs that include a peptide containing an amino acid sequence that includes a cleavage site specific for FAP or an enzyme having a proteolytic activity of FAP. Cleavage sites and activity for FAP have been described, for example, in U.S. patent application Ser. Nos. 12/087,398 and 13/471,316, and FAP-activated anti-tumor compounds have been described in U.S. patent application Ser. Nos. 10/039,781, 10/036,111, 10/336,378, and 10/036,224, and U.S. Pat. No. 6,613,879.

In one embodiment, the agents that substrates of FAP comprise include a peptide that comprises the sequence VGPAGK (SEQ ID NO.: 172); GARGQA (SEQ ID NO.: 173); PPGPPGPA (SEQ ID NO.: 174); (D/E)RG(E/A)(T/S)GPA (SEQ ID NO:175); DRGETGPA (SEQ ID NO: 176); RTGDAGPA (SEQ ID NO: 177); ASGPAGPA (SEQ ID NO: 178); DRGETGPA (SEQ ID NO: 179); DKGESGPA (SEQ ID NO: 180); AKGEAGPA (SEQ ID NO:181); PPGPPGPA (SEQ ID NO: 182); EPGPPGPA (SEQ ID NO: 183); DAGPPGPA (SEQ ID NO: 184); GETGPAGA (SEQ ID NO: 185); QPSGPAGA (SEQ ID NO: 186); ERGETGPA (SEQ ID NO: 187); DRGATGPA (SEQ ID NO: 188); DRGESGPA (SEQ ID NO: 189); DPGETGPA (SEQ ID NO: 190); LNGLPGA (SEQ ID NO: 191); PSGPAGPA (SEQ ID NO: 192); PAGAAGPA (SEQ ID NO: 193); FPGARGPA (SEQ ID NO: 194); FQGLPGPA (SEQ ID NO: 195); PLGAPGPA (SEQ ID NO: 196); PPGAVGPA (SEQ ID NO: 197); MGFPGPA (SEQ ID NO: 198); RVGPPGPA (SEQ ID NO: 199); AGPVGPPA (SEQ ID NO: 200); AGPPGPPA (SEQ ID NO: 201); EPGASGPA (SEQ ID NO: 202); ETGPAGPA (SEQ ID NO: 203); PPGAVGPA (SEQ ID NO: 204); AQGPPGPA (SEQ ID NO: 205); KTGPPGPA (SEQ ID NO: 206); VMGFPGPA (SEQ ID NO: 207); or SGEAGPA (SEQ ID NO: 208), and portions and variants thereof.

In another embodiment, the substrates of FAP comprise a peptide that comprises the sequence XXXXX-A (SEQ ID NO: 209); XXXX-AG (SEQ ID NO: 210); XXXX-AGG (SEQ ID NO: 211); XXXXX-AGG (SEQ ID NO: 482); XXXX-S (SEQ ID NO: 212]; XXXX-SG (SEQ ID NO: 213); XXXX-V (SEQ ID NO: 214); or XXXXVG (SEQ ID NO: 215), wherein X is any amino acid, and portions and variants thereof.

Other peptide substrates of FAP falling within the scope of the invention include peptides with prolines as most cleavage by FAP has been observed after Pro. Other peptides may contain the following amino acids as FAP was found to cleave after: Ala (e.g. A/A, A/G, A/P, A/R), Asp (e.g., D/G, D/T), Gly (e.g., G/A, G/E, G/L, G/Q, G/P, G/V), Glu (e.g., E/P), Lys (e.g., K/A, K/G), Ser (e.g., S/P) and Val (e.g., V/G).

Other embodiments include FAP substrate peptides with varying lengths in the P′ positions (e.g., PI-P′3). That is, sequences with proline in P1 but having either nothing in P1, Ala, Ser, Val in P1, or Ala, Ser Val in PI and Gly in P′2.

Other peptides would have the following sequences for FAP, showing a preference for Asp or Glu, Arg or Ala residues in P7, Arg or Lys in P6, Ala, Asp or Glu in P4, Ala, Ser or Thr in P3 and Ala, Ser or Val in P′1 and Gly in P′2.

FAP-selective cleavage sites were identified using methods described in U.S. patent application Ser. No. 12/087,398. FAP cleavage sites that were identified within the 8.5 kDa fragment of recombinant human gelatin are shown in Table 2.

TABLE 2
FAP cleavage sites (P7-P′3) within the
8.5 kDa fragment of recombinant human gelatin

| | | Normalized |
|---|---|---|
| Cleavage Fragment | MH+ | Ion Current |
| PPGAVGP/AGK . . . AQGPPGP/AGP | 1308.62 | 234.8 |
| (SEQ ID NOS: 221 and 237) | | |
| --GLP . . . SPGSPGP/DGK | 1449.77 | 76.3 |
| (SEQ ID NO: 238) | | |
| KTGPPGP/AGQ . . . PPGPPGA/RGQ | 1330.65 | 57.2 |
| (SEQ ID NOS: 222 and 239) | | |
| VMGFPGP/ KGA . . . PPGAVGP/AGK | 2113.12 | 46.8 |
| (SEQ ID NOS: 223 and 240) | | |
| VMGFPGP/KGA . . . GEPGKAG/ERG | 942.5 | 14.7 |
| (SEQ ID NOS: 224 and 241) | | |
| --GLP . . . KTGPPGP/AGQ | 2256.16 | 12.7 |
| (SEQ ID NO: 242) | | |
| GFPGPKG/AAG . . . PPGAVGP/AGK | 1928 | 10.1 |
| (SEQ ID NOS: 225 and 243) | | |
| FPGPKGA/AGE . . . PPGAVGP/AGK | 1856.96 | 6.6 |
| (SEQ ID NOS: 226 and 244) | | |
| LTGSPGS/PGP . . . KTGPPGP/AGQ | 1076.54 | 6.0 |
| (SEQ ID NOS: 227 and 245) | | |
| KTGPPGP/AGQ . . . GPPGPPG/ARG | 1259.61 | 5.0 |
| (SEQ ID NOS: 228 and 246) | | |
| VMGFPGP/KGA . . . AGEPGKA/GER | 885.48 | 4.7 |
| (SEQ ID NOS: 229 and 247) | | |
| GLPGAKG/LTG . . . SPGSPGP/DGK | 869.44 | 2.7 |
| (SEQ ID NOS: 230 and 248) | | |
| MGFPGPK/GA-AGEPGKA/GER | 757.38 | 2.5 |
| (SEQ ID NOS: 231 and 249) | | |
| PGPPGAR/GQA . . . VMGFPGP KGA | 1017.48 | 2.4 |
| (SEQ ID NOS: 232 and 250) | | |
| PGARGQA/G-VMGFPGP/KGA | 761.37 | 1.7 |
| (SEQ ID NOS: 233 and 251) | | |
| GPPGPPG/ARG . . . VMGFPGP/KGA | 1244.62 | 1.7 |
| (SEQ ID NOS: 234 and 252) | | |
| PPGPPGA/RGQ . . . VMGFPGP/KGA | 1173.58 | 1.3 |
| (SEQ ID NOS: 235 and 253) | | |
| KTGPPGP/AGQ . . . GARGQAG/VMG | 1799.89 | 1.0 |
| (SEQ ID NOS: 236 and 254) | | |

Analysis of data in Table 2 demonstrate that FAP cleavage primarily occurred after proline (P), but FAP could also cleave after other amino acids including glycine (G), alanine (A), lysine (K) and arginine (R) (underlined sequences in Table 2). Based on normalized ion current, it appeared that more abundant ions consisted of those with proline as cleavage site. In those sequences, G was the preferred amino acid in the P2 position.

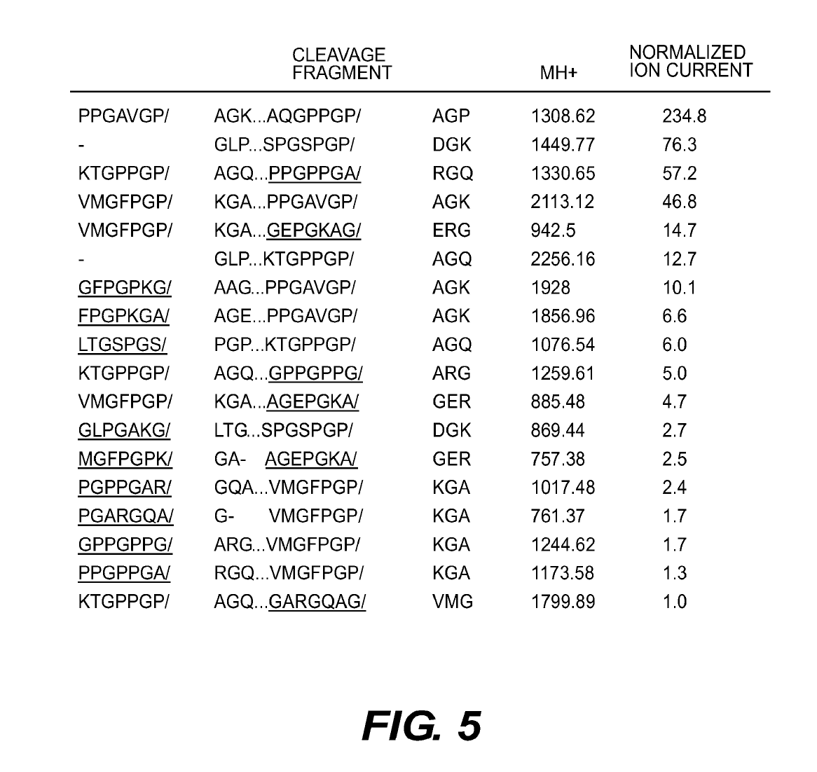

On the basis of the 8.5 kDa gelatin cleavage map a series of fluorescence quenched substrates were prepared (colored sequences in Table 2) by using the Methoxycoumarin (MCA)/Dinitrophenyl (DNP) FRET combination. See, e.g., Matsushita, O., et al. J. Bacteriol, 176, 149-156 (1994). Synthesis of peptides was done using standard Fmoc solid phase peptide synthesis coupling on a NovaTag™ Dnp resin with a substitution level of 0.4 mmole/g (Novabiochem, San Diego, Calif.). N-terminal capping was done twice overnight with N-(7-methoxycoumarin-4-acetyloxy)succinimide (MCA-Osu) and 1-Hydroxybenzotriazole (HOBt) in N-Methyl 2 Pyrrolidone (NMP). This synthetic method yields peptides with sequence MCA-AA1-AA2-AA3-AAx-DNP. These studies supported substrate ranking based on normalized ion current and confirmed that FAP prefers to cleave after proline but can also cleave after other amino acids in the P1 position, FIG. 5. The results also suggest that FAP hydrolysis increases with increasing number of amino acids in the P′ positions with VGP//AGK cleavage>GAVGP//A>PAGP//(SEQ ID NOS: 464, 466, and 468, respectively), FIG. 5.

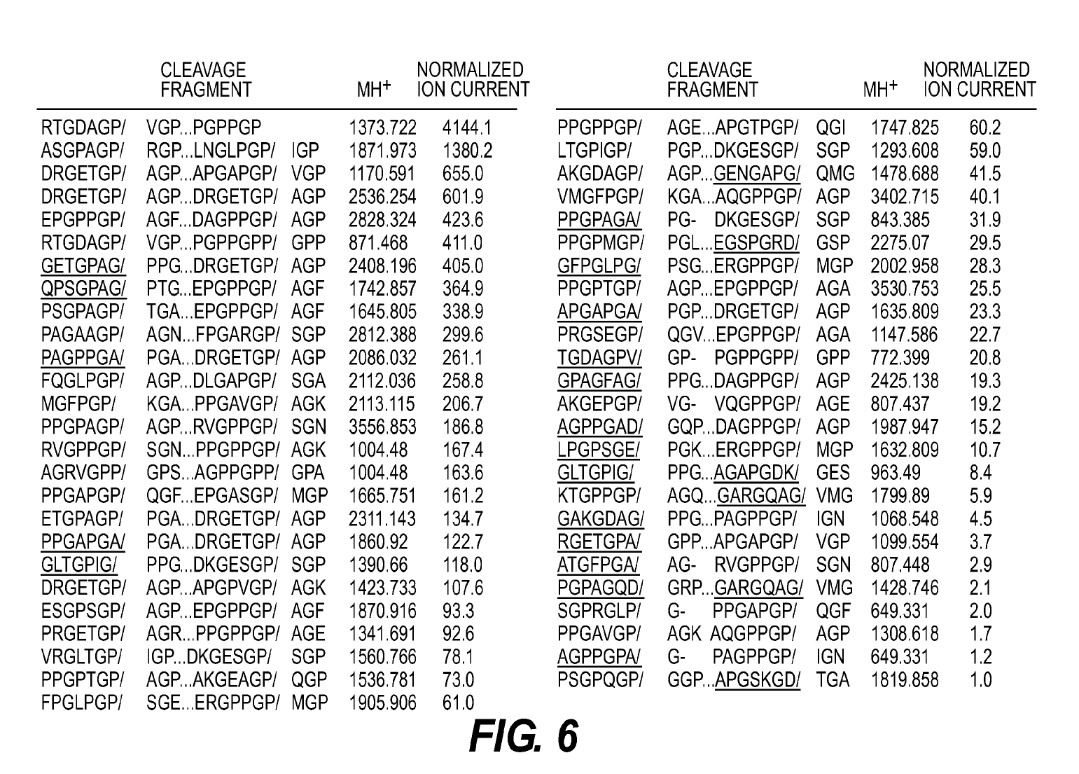

Additional cleavage sites were identified and shown in Tables 3 and 4, and a complete map of FAP cleavage sites within a 100 kDa gelatin produced by FibroGen is shown in FIG. 6.

TABLE 3
FindPept alignment of MALDI masses for digest of 8.5 KDa Gelatin

| | | Δmass | | SEQ | | Missed |
|---|---|---|---|---|---|---|
| User mass | DB mass | (daltons) | peptide | ID NO: | position | cleavages |
| 2144.530 | 2113.078 | −1.451 | (K) GAAGEPGKAGERGVPGPPGA | 255 | 55-79 | 0 |
| | | | VGPAG (K) | | | |
| 2114.530 | 2113.115 | −1.415 | (P) GPKGAAGEPGKAGERGVPGP | 256 | 52-75 | 0 |
| | | | PGAV (G) | | | |
| 2114.530 | 2113.11 | −1.415 | (G) PKGAAGEPGKAGERGVPGPP | 257 | 53-76 | 0 |
| | | | GAVG (P) | | | |
| 2114.530 | 2113.115 | −1.415 | (P) KGAAGEPGKAGERGVPGPPG | 258 | 54-77 | 0 |
| | | | AVGP (A) | | | |
| 2114.530 | 2113.115 | −1.415 | (A) AGEPGKAGERGVPGPPGAVG | 259 | 57-80 | 0 |
| | | | PAGK (D) | | | |
| 2114.530 | 2114.071 | −0.458 | (D) GRPGPPGPPGARGQAGVMGF | 260 | 31-53 | 0 |
| | | | PGP (K) | | | |
| 2114.530 | 2115.010 | 0.480 | (G) AVGPAGKDGEAGAQGPPGPA | 261 | 74-98 | 0 |
| | | | GPAGE R | | | |
| 2449.780 | 2448.245 | −1.534 | (Q) AGVMGFPGPKGAAGEPGKAG | 262 | 45-71 | 0 |
| | | | ERGVPGP (P) | | | |
| 2449.780 | 2451.165 | 1.384 | (T) GSPGSPGPDGKTGPPGPAGQ | 263 | 10-37 | 0 |
| | | | DGRPGPPG (P) | | | |
| 2449.780 | 2451.220 | 1.439 | (P) PGARGQAGVMGFPGPKGAAG | 264 | 39-65 | 0 |
| | | | EPGKAGE R | | | |
| 3402.320 | 3402.678 | 0.358 | (K) GAAGEPGKAGERGVPGPPGA | 265 | 55-94 | 0 |
| | | | VGPAGKDGEAGAQGPPGPAG (P) | | | |
| 3402.320 | 3402.715 | 0.394 | (F) PGPKGAAGEPGKAGERGVPG | 266 | 51-89 | 0 |
| | | | PPGAVGPAGKDGEAGAQGP (P) | | | |
| 3402.320 | 3402.715 | 0.394 | (P) GPKGAAGEPGKAGERGVPGP | 267 | 52-90 | 0 |
| | | | PGAVGPAGKDGEAGAQGPP (G) | | | |
| 3402.320 | 3402.715 | 0.394 | (G) PKGAAGEPGKAGERGVPGPP | 268 | 53-91 | 0 |
| | | | GAVGPAGKDGEAGAQGPPG (P) | | | |
| 3402.320 | 3402.715 | 0.394 | (P) KGAAGEPGKAGERGVPGPPG | 269 | 54-92 | 0 |
| | | | AVGPAGKDGEAGAQGPPGP (A) | | | |
| 3402.320 | 3403.671 | 1.351 | (T) GPPGPAGQDGRPGPPGPPGA | 270 | 22-59 | 0 |
| | | | RGQAGVMGFPGPKGAAGE (P) | | | |
| 3402.320 | 3403.671 | 1.351 | (P) PGPAGQDGRPGPPGPPGARG | 483 | 24-61 | 0 |
| | | | QAGVMGFPGPKGAAGEPG (K) | | | |

TABLE 4
Full map of FAP cleavage sites within 100 kDa human gelatinase

| | | | SEQ | | | Per- |
|---|---|---|---|---|---|---|
| P6- | | P′2- | ID | | Occur- | cent- |
| P2 | Sequence | P′4 | NO: | Mass | rences | age (%) |
| TGFPG | A.AGRVGPPGP . S | GNA | 272 | 807.448 | 2 | 0.95 |
| GPPGP | A.GPAGPPGP . I | GNV | 273 | 649.331 | 1 | 0.48 |
| GETGP | A.GPPGAPGAPGAPGP .V | GPA | 274 | 1099.554 | 1 | 0.48 |
| AGPPG | A.PGAPGAPGPVGPAGKSGDRG | GPA | 275 | 2086.032 | 4 | 1.90 |
| | ETGP . A | | | | | |
| PGAPG | A.PGAPGPVGPAGKSGDRGETG | GPA | 276 | 1860.92 | 2 | 0.95 |
| | P .A | | | | | |
| PGPAG | A.PGDKGESGP .S | GPA | 277 | 843.385 | 2 | 0.95 |
| PGAPG | A.PGPVGPAGKSGDRGETGP .A | GPA | 278 | 1635.809 | 1 | 0.48 |
| GPPGA | D.GQPGAKGEPGDAGAKGDAG | GPA | 279 | 1987.947 | 2 | 0.95 |
| | PPGP .A | | | | | |
| GPAGQ | D.GRPGPPGPPGARGQAG .V | MGF | 280 | 1428.746 | 2 | 0.95 |
| PGPSG | E.PGKQGPSGASGERGPPGP .M | GPP | 281 | 1632.809 | 5 | 2.38 |
| PAGFA | G.PPGADGQPGAKGEPGDAGAK | GPA | 282 | 2425.138 | 2 | 0.95 |
| | GDAGPPGP .A | | | | | |
| ETGPA | G.PPGAPGAPGAPGPVGPAGKS | GPA | 283 | 2408.196 | 7 | 3.33 |
| | GDRGETGP .A | | | | | |
| LTGPI | G.PPGPAGAPGDK .G | ESG | 284 | 963.49 | 2 | 0.95 |
| LTGPI | G.PPGPAGAPGDKGESGP .S | GPA | 285 | 1390.66 | 3 | 1.43 |
| AKGDA | G.PPGPAGPAGPPGP .I | GNV | 286 | 1068.548 | 1 | 0.48 |
| FPGLP | G.PSGEPGKQGPSGASGERGPPG | GPP | 287 | 2002.958 | 1 | 0.48 |
| | P .M | | | | | |
| PSGPA | G.PTGARGAPGDRGEPGPPGP .A | GFA | 288 | 1742.857 | 6 | 2.86 |
| PGPPG | P.AGEKGSPGADGPAGAPGTPG | GIA | 289 | 1747.825 | 7 | 3.33 |
| | P .Q | | | | | |
| PGPPG | P.AGFAGPPGADGQPGAKGEPG | GPA | 290 | 2828.324 | 8 | 3.81 |
| | DAGAKGDAGPPGP .A | | | | | |
| PGAVG | P.AGKDGEAGAQGPPGP .A | GPA | 291 | 1308.618 | 2 | 0.95 |
| AGAAG | P.AGNPGADGQPGAKGANGAP | GPQ | 292 | 2812.388 | 4 | 1.90 |
| | GIAGAPGFPGARGP .S | | | | | |
| KGDAG | P.AGPKGEPGSPGENGAPG .Q | MGP | 293 | 1478.688 | 1 | 0.48 |
| RGETG | P.AGPPGAPGAPGAPGP .V | GPA | 294 | 1170.591 | 8 | 3.81 |
| RGETG | P.AGPPGAPGAPGAPGPVGP .A | GKS | 295 | 1423.733 | 2 | 0.95 |
| RGETG | P.AGPPGAPGAPGAPGPVGPAG | GPA | 296 | 2536.254 | 6 | 2.86 |
| | KSGDRGETGP .A | | | | | |
| QGLPG | P.AGPPGEAGKPGEQGVPGDLG | GAR | 297 | 2112.036 | 6 | 2.86 |
| | APGP .S | | | | | |
| PGPTG | P.AGPPGFPGAVGAKGEAGP .Q | GPR | 298 | 1536.781 | 2 | 0.95 |
| PGPTG | P.AGPPGFPGAVGAKGEAGPQG | GAA | 299 | 3530.753 | 1 | 0.48 |
| | PRGSEGPQGVRGEPGPPGP .A | | | | | |
| PGPAG | P.AGPPGPIGNVGAPGAKGARG | GNA | 300 | 3556.853 | 3 | 1.43 |
| | SAGPPGATGFPGAAGRVGPPGP .S | | | | | |
| SGPSG | P.AGPTGARGAPGDRGEPGPPGP. | GFA | 301 | 1870.916 | 6 | 2.86 |
| | A | | | | | |
| TGPPG | P.AGQDGRPGPPGPPGARGQAG .V | MCF | 302 | 1799.89 | 1 | 0.48 |
| RGETG | P.AGRPGEVGPPGPPGP .A | GEK | 303 | 1341.691 | 1 | 5.24 |
| SGPQG | P.GGPPGPKGNSGEPGAPGSKG | GAK | 304 | 1819.858 | 1 | 0.48 |
| | D .T | | | | | |
| GPRGL | P.GPPGAPGP .Q | GFQ | 484 | 649.331 | 1 | 0.48 |
| GRVGP | P.GPSGNAGPPGPP .G | PAG | 306 | 1004.48 | 1 | 0.48 |
| RGLTG | P.IGPPGPAGAPGDKGESGP .S | GPA | 307 | 1560.766 | 6 | 2.86 |
| MGFPG | P.KGAAGEPGKAGERGVPGPPG | GKD | 308 | 2113.115 | 5 | 2.38 |
| | AVGP .A | | | | | |
| MGFPG | P.KGAAGEPGKAGERGVPGPPG | GPA | 309 | 3402.715 | 1 | 0.48 |
| | AVGPAGKDGEAGAQGPPGP .A | | | | | |
| TGPAG | P.PGAPGAPGAPGPVGPAGKSG | GPA | 310 | 2311.143 | 2 | 0.95 |
| | DRGETGP .A | | | | | |
| PGPMG | P.PGLAGPPGESGREGAPGAEGS | SPG | 311 | 2275.07 | 2 | 0.95 |
| | PGRD .G | | | | | |
| TGPIG | P.PGPAGAPGDKGESGP .S | GPA | 312 | 1293.608 | 2 | 0.95 |
| PGAPG | P.QGFQGPPGEPGEPGASGP .M | GPR | 313 | 1685.751 | 2 | 0.95 |
| RGSEG | P.QGVRGEPGPPGP .A | GAA | 314 | 1147.585 | 3 | 1.43 |
| SGPAG | P.RGPPGSAGAPGKDGLNGLPG | GPP | 315 | 1871.973 | 14 | 6.67 |
| | P .I | | | | | |
| PGLPG | P.SGEPGKQGPSGASGERGPPGP . | GPP | 316 | 1905.906 | 4 | 1.90 |
| | M | | | | | |
| VGPPG | P.SGNAGPPGPPGP .A | GKE | 317 | 1004.48 | 14 | 6.67 |
| SGPAG | P.TGARGAPGDRGEPGPPGP .A | GFA | 318 | 1645.805 | 13 | 6.19 |
| TGDAG | P.VGPPGPPGPP .G | PPG | 319 | 671.468 | 3 | 1.43 |
| TGDAG | P.VGPPGPPGPPGPPGPP . | | 320 | 1373.722 | 9 | 9.05 |
| KGEPG | P.VGVQGPPGP .A | GEE | 321 | 807.437 | 3 | 1.43 |
| GDAGP | V.GPPGPPGPP .G | PPG | 322 | 772.399 | 2 | 0.95 |

The prodrugs of the invention are not taken up by the cells, but are cleaved extracelullarly by the specific targeted protease (for example, PSMA, PSA, hK2 or FAP) to yield at least 5 picomoles, preferably at least 10 picomoles, and more preferably at least 15 picomoles of therapeutic drug per minute per milligram of the specific protease. Preferably, the prodrugs of the invention are cleaved by extracellular proteases other than the specific targeted protease (e.g., PSMA, PSA, hK2 or FAP) to yield not more than 4.0 picomoles, preferably not more than 2.0 picomoles, and more preferably not more than 1.0 picomole of therapeutic drug per minute per milligram of purified extracellular non-specific proteases (i.e. any protease other than the specific targeted protease).

Thus, for example, a peptide prodrug of the invention that is cleavable by PSMA is not taken up by the cells, but is cleaved extracelullarly by PSMA to yield at least 5 picomoles, preferably at least 10 picomoles, and more preferably at least 15 picomoles of therapeutic drug per minute per milligram of PSMA. Preferably, the PSMA-specific prodrugs of the invention are cleaved by extracellular proteases other than PSMA to yield not more than 4.0 picomoles, preferably not more than 2.0 picomoles, and more preferably not more than 1.0 picomole of therapeutic drug per minute per milligram of purified extracellular non-PSMA proteases

The prodrugs of the invention yield at most 5%, preferably at most 2.5%, and more preferably at most 1.0% of prodrug as therapeutic drug in human serum over a 24-hour period.

The peptides of the present invention may be synthesized by methods known in the art. For example, peptides and prodrugs of the invention may be synthesized by the methods of U.S. Pat. Nos. 6,632,922; 6,649,136; 6,310,180; 4,749,742 and U.S. Application No. 61/791,909. Peptides may also be synthesized on automated peptide synthesizing machines (e.g., the Symphony/Multiplex™ automated peptide synthesizer (Protein Technologies, Inc, Tucson, Ariz.) or the Perkin-Elmer (Applied Biosystems, Foster City, Calif.) Model 433A automated peptide synthesizer).

Deletion of one or more amino acids can also result in a modification of the structure of the resultant molecule without significantly altering its biological activity. This can lead to the development of a smaller active molecule which would also have utility. For example, amino or carboxy terminal amino acids which may not be required for biological activity of the particular peptide can be removed. Peptides of the invention include any analog, homolog, mutant, isomer or derivative of the peptides disclosed in the present invention, as long as the bioactivity as described herein remains. The peptides described in one embodiment have sequences comprised of L-amino acids; however, D-forms of the amino acids can be synthetically produced and used in the peptides described herein. In yet another embodiment, the amino acids are non-naturally occurring amino acids, which are known to one of skill in the art.

The peptides of the invention include peptides which are conservative variations of those peptides specifically exemplified herein. Conservative variations may also include the use of a substituted amino acid in place of an unsubstituted parent amino acid provided that antibodies raised to the substituted polypeptide also immunoreact with the unsubstituted polypeptide. Such conservative amino acid substitutions are within the definition of the classes of the peptides of the invention with respect to X positions which may be any of a number of amino acids. The peptides which are produced by such conservative variations can be screened for suitability of use in the prodrugs of the invention according to the methods for selecting prodrugs provided herein.

Further examples of the peptides of the invention are constructed as analogs of, derivatives of, and conservative variations on the amino acids sequences disclosed herein. Thus, the broader group of peptides having hydrophilic and hydrophobic substitutions, and conservative variations are encompassed by the invention. Those of skill in the art can make similar substitutions to achieve peptides with greater activity and/or specificity toward the proteases described herein. For example, the invention includes the peptide sequences described above, as well as analogs or derivatives thereof, as long as the bioactivity of the peptide remains. Minor modifications of the primary amino acid sequence of the peptides of the invention may result in peptides which have substantially equivalent activity as compared to the specific peptides described herein. Such modifications may be deliberate, as by site-directed mutagenesis or chemical synthesis, or may be spontaneous. All of the peptides produced by these modifications are included herein, as long as the biological activity of the original peptide remains, i.e., susceptibility to cleavage by a selected protease, for example, PSMA, PSA, hK2 or FAP.

In one embodiment, a wide variety of groups can be linked to the carboxy terminus of the peptides disclosed herein, for example, with peptides that are susceptible to cleavage by PSA, hK2 and FAP. See, for example, U.S. Pat. Nos. 7,906,477, 7,053,042, 6,265,540, 6,410,514, 6,504,014, 6,545,131, 7,635,682, and U.S. patent application Ser. Nos. 12/087,398 and 13/471,316. Notably, therapeutic drugs can be linked to this position.

In another embodiment, for example, with peptides that are susceptible to cleavage but PSMA, a wide variety of entities, including therapeutic drugs, can be linked to the α-amino terminus, the α-carboxy terminus, or a side chain of the peptide, as described in U.S. Pat. Nos. 7,767,648 and 7,468,354. A further preferred embodiment in this embodiment, the linkage between the entity (e.g., the therapeutic drug) and the peptide takes place at either the amino terminus, or at the side chain of the peptide. In one example of this embodiment, with a PSMA prodrug of the invention the entity is linked preferably at X₁, however, the entity may be linked at any position from X₁ to Xₙ₋₁.

In both of the above embodiments, advantage is taken of the protease-specificity of the cleavage site, as well as other functional characteristics of the peptides of the invention. Preferably, the therapeutic drugs are linked to either the carboxy terminus or the α-amino terminus, as applicable, either directly or through a linker group. The direct linkage is preferably through an amide bond, in order to utilize the proteolytic activity and specificity of the protease. If the connection between the therapeutic drug and the amino acid sequence is made through a linker, this connection is also preferably made through an amide bond, for the same reason. The linker may be connected to the therapeutic drug through any of the bond types and chemical groups known to those skilled in the art. The linker may remain on the therapeutic drug indefinitely after cleavage, or may be removed soon thereafter, either by further reactions with external agents, or in a self-cleaving step. Self-cleaving linkers are those linkers that can intramolecularly cyclize and release the drug, or undergo spontaneous S_{N1} solvolysis and release the drug upon peptide cleavage.

Other materials such as detectable labels or imaging compounds can be linked to the peptide. Various groups that may also be linked to the peptide include such moieties as antibodies, and peptide toxins, including the 26 amino acid toxin melittin and the 35 amino acid toxin cecropin B, for example. Both of these peptide toxins have shown toxicity against cancer cell lines. Additionally, the peptide may be coupled to a protein. This coupling can be used to create an inactive proenzyme so that cleavage by the selected protease would cause a conformational change in the protein to activate it. The peptide sequence may also be used to couple a drug to an antibody. The antibody would bind to a cell surface protein and tissue-specific protease present in the extracellular fluid could cleave the drug from the peptide linker. See, for example, U.S. Pat. Nos. 7,906,477 and 7,053,042, and U.S. application Ser. Nos. 12/087,398 and 13/471,316.

The preferred amino acid sequence can be constructed to be highly specific for cleavage by a selected protease of the invention, for example, PSMA, PSA, FAP or hK2. In addition the peptide sequence can be constructed to be highly selective towards cleavage by a selected protease as compared to purified extracellular and intracellular proteases. Highly-specific protease-specific peptide sequences can also be constructed that are also stable toward cleavage in human sera. Methods of constructing and selecting substrates of the invention, for example, FAP-, PSMA-, PSA- or hK2-substrates, are known in the art and disclosed herein.

In some embodiments, the present invention contemplates that the peptides of the present invention are protease-resistant and degradation resistant. Such embodiments of the peptides of the present invention and peptides comprising various protecting groups are described in U.S. patent application Ser. Nos. 12/087,398 and 13/471,316.

Labeling and Screening of Peptides and Substrates

Procedures for labeling peptides that are protease-specific substrates are described, for example, in U.S. patent application Ser. Nos. 12/087,398 and 13/471,316. Further, methods for selecting potential prodrugs for use in the present invention and methods for screening tissue and determining the activity of a selected protease are known in the art.

Imaging and Diagnostic Applications

In one embodiment, the injectable emulsions described herein may be used to administer the active compounds of the invention for imaging and diagnosing a cell proliferative disorder or cancer in a subject suspected of having cancer, a subject diagnosed with a cancer or a subject at risk for having cancer. Methods for diagnosing and imaging a cell proliferative disorder or cancer in a patient using the active compounds of the invention are described in U.S. Application. No. 13/257,131.

In one embodiment, the injectable emulsions described herein may be used to administer the active compounds of the invention for imaging and diagnosing a cell proliferative disorder, including cancer, in a subject that is suspected of having, has been diagnosed with or is at risk for carcinoma, melanoma, lymphoma, blastoma, sarcoma, and leukemia or lymphoid malignancy. Further, in this embodiment, the injectable emulsions described herein may be used to administer the active compounds of the invention for imaging and diagnosing cancer in a subject that has been diagnosed with or is at risk for epithelial cancers. In a further variation of this embodiment, the injectable emulsions described herein may be used to administer the active compounds of the invention for imaging and diagnosing cancer in a subject that has been diagnosed with or is at risk for prostate, liver or breast cancer. In another embodiment, the injectable emulsions described herein may be used to administer the active compounds of the invention for imaging and diagnosing a cell proliferative disorder, for example, BPH.

In one embodiment, the active compounds of the invention comprise a phenolic linker between the therapeutic drug and the peptide. The phenolic linker may be radiolabeled. In certain embodiments, the radiolabel is ¹²⁵I, ¹²⁴I or ¹³¹I. Further, in other aspects of this embodiment, the short range of alpha or beta irradiation makes labeling with alpha or beta emitters advantageous to gamma emitters, such as the iodine radiolabels. Tritium (³H) is a representative beta emitter suitable for use with the presently disclosed methods and compositions. The peptide of the present invention may be any peptide that is cleavable by a selected protease, for example, PSMA, PSA, hK2 and FAP.

In further aspects of this embodiment, the therapeutic drug is a sesquiterpene-γ-lactone (for example, a thapsigargin or thapsigargin derivative).

In one alternative of these embodiments of the invention, methods of imaging a subject include the use of single photon emission computed tomography (SPECT) imaging. Still in another alternative of these embodiments, the imaging may be positron emission tomography (PET). For example, the phenolic linker can be labeled with ¹²⁵I for SPECT imaging, ¹²⁴I for PET imaging, and ¹³¹I for combination drug/radiation therapy.

In a further embodiment, methods of imaging and diagnosing a cell proliferative disorder, including cancer, may include providing to a subject an injectable emulsion of the invention comprising a prodrug comprising a sesquiterpene-γ-lactone (in some preferred embodiments, thapsigargin or a thapsigargin derivative), a phenolic linker with a radiolabel and a peptide cleavable by PSA, FAP, a hK2 or PSMA, or any other peptide that may be cleaved by a selected protease.

In one variation of this embodiment, the thapsigargin derivative is 8-O-(12-[L-leucinoylamino] dodecanoyl)8-O-debutanoylthapsigargin (L12ADT). In another variation of this embodiment, the thapsigargin derivative is 8-O-(12-aminododecanoyl)-8-O-debutanoyl thapsigargin (12ADT).

The invention also provides a method of imaging soft tissue or bone metastases by providing peptides of the invention linked to lipophilic imaging labels that can be detected by imaging techniques, for example, PET. This method is accomplished generally by administering a peptide of the invention linked to a primary amine-containing lipophilic label to a subject having or suspected of having a cell proliferative disorder associated with a protease capable of cleaving a peptide of the invention, for example, PSA, PSMA, hK2 or FAP. In a preferred embodiment, the labeled peptide is administered using the injectable emulsions of the invention and coupled to a therapeutic drug, such as a sesquiterpene-γ-lactone (in some preferred embodiments, thapsigargin or a thapsigargin derivative). The peptide is selectively cleaved from the lipophilic imaging label where there is enzymatically active protease specific to the peptide to be cleaved (for example, PSMA, PSA, hK2 or FAP producing tissues). The lipophilic imaging label is then drawn into the membranes of cells in the vicinity.

After a period of time sufficient to allow cleavage of the peptide by the selected protease, and to allow the uncleaved peptide to be sufficiently cleared from the subject to allow reliable imaging, the subject is imaged. The lipophilic label accumulates in the soft tissue or bone that produces the selected protease, and allows a diagnosis of the subject. Suitable labels for PET scanning are radionuclides, such ¹⁸F, ¹¹C, ¹³N and, ¹⁵O, and any other positron emitters known in the art. Lipophilicity can be engineered into the label by introducing the label into lipophilic fragments or moieties known to those in the art, by methods known to those skilled in the art.

Examples of various linkers that may be used with the peptide prodrugs of the present invention for imaging and diagnostic applications are disclosed in U.S. application Ser. No. 13/257,131. Examples of methods of making compositions for the imaging and detection of cancer using the peptide prodrugs of the present invention are also disclosed therein and generally known in the art.

Prodrug Compositions

The invention also features prodrug compositions that comprise a therapeutic drug linked to a peptide containing a cleavage site that is specific for a selected protease, for example, FAP, PSMA, PSA and hK2, or any enzyme that has the enzymatic activity of such a protease. As noted above, the peptides of the invention can be used to target therapeutic drugs for activation at or within tissue or cells producing a selected protease, or tumor-associated cells. The peptides that are useful in the prodrugs of the invention are those described above.

The therapeutic drugs that may be used in the prodrugs of the invention include, for example, sesquiterpene-γ-lactones such as those belonging to the guaianolide, inuchineolide, germacranolide, and eudesmanolide families of scsquitcrpcnoids. These include estafiatin, grosshcimin, inuchincnolidc, arglabin, thapsigargin and their derivatives, such as thapsigargicin and many others known to those skilled in the art. See, e.g., U.S. Pat. No. 6,545,131.

In one embodiment, a preferred sesquiterpene-γ-lactone of the present invention is thapsigargin or a thapsigargin derivative. As stated above, thapsigargin and its derivatives are believed to act by inhibiting the SERCA pump found in many cells. The thapsigargins are a group of natural products isolated from species of the umbelliferous genus Thapsia.

Thapsigargin has a unique mechanism of cytotoxicity. Without wishing to be bound by an particular scientific theory, it is a potent inhibitor of the Sarcoplasmic/Endoplasrnic Reticulum Calcium ATPase pump which is a critical intracellular protein required by all cells to maintain metabolic viability. Inhibition of the SERCA pump by thapsigargin leads to sustained elevation of intracellular calcium which activates both ER-stress and mitochondrial apoptotic pathways.

Thapsigargin has the structure disclosed in U.S. Pat. No. 6,545,131. In addition, other therapeutic analogs of sesquitepene-γ-lactones are also disclosed in U.S. Pat. Nos. 6,265,540, 6,504,014 6,410,514, and 6,545,131. These analogs have non-specific toxicity toward cells. This toxicity is measured as the toxicity needed to kill 50% of clonogenic cells (LC₅₀). In some embodiments, the LC₅₀ of the analogs of this invention is desirably at most 10 μM, preferably at most 2 μM and more preferably at most 200 nM of analog.

In one example, thapsigargins with alkanoyl, alkenoyl, and arenoyl groups at carbon 8 or carbon 2, can be employed in the practice of the invention disclosed herein. Groups such as CO—(CH══CH)ₙ₁—(CH2)ₙ₂-Ar—NH₂, CO—(CH₂)ₙ₂—(CH══CH)ₙ₁—Ar—NH₂, CO—(CH2)ₙ₂-(CH══CH)ₙ₁—CO—NH—Ar—NH₂ and CO—(CH══CH)ₙ₁—(CH2)ₙ₂-CO—NH—Ar—NH₂ and substituted variations thereof can be used as carbon 8 substituents, where n1 and n2 are from 0 to 5, and Ar is any substituted or unsubstituted aryl group. Substituents which may be present on Ar include short and medium chain alkyl, alkanoxy, aryl, aryloxy, and alkenoxy groups, nitro, halo, and primary secondary or tertiary amino groups, as well as such groups connected to Ar by ester or amide linkages.

In other embodiments of thapsigargin analogs, these substituent groups are represented by unsubstituted, or alkyl-, aryl-, halo-, alkoxy-, alkenyl-, amino-, or amino-substituted CO—(CH₂)ₙ₃—NH2, where n3 is from 0 to 15, preferably 3-15, and also preferably 6-12. Particularly preferred substituent groups within this class are 6-aminohexanoyl, 7-aminoheptanoyl, 8-aminooctanoyl, 9-aminononanoyl, 10-aminodecanoyl, 11-aminoundecanoyl, and 12-aminododecanoyl. These substituents are generally synthesized from the corresponding amino acids, 6-aminohexanoic acid, and so forth. The amino acids are N-terminal protected by standard methods, for example Boc protection. Dicyclohexylcarbodiimide (DCCI)-promoted coupling of the N-terminal protected substituent to thapsigargin, followed by standard deprotection reactions produces primary amine-containing thapsigargin analogs.

The substituents can also carry primary amines in the form of an amino amide group attached to the alkanoyl-, alkenyl-, or arenoyl substituents. For example, C-terminal protection of a first amino acid such as 6-aminohexanoic acid and the like, by standard C-terminal protection techniques such as methyl ester formation by treatment with methanol and thionyl chloride, can be followed by coupling the N-terminal of the first amino acid with an N-protected second amino acid of any type.

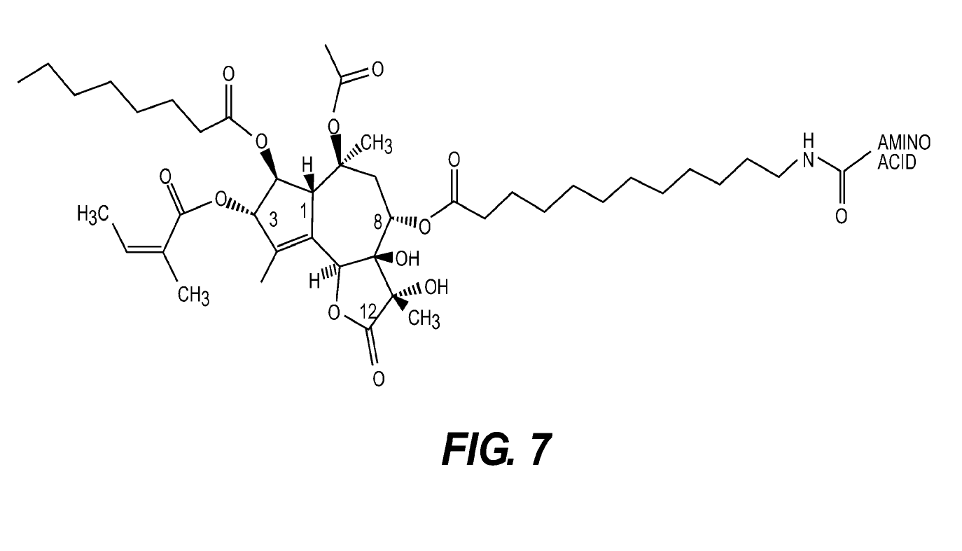

In a preferred embodiment, the thapsigargin analog or derivative is 8-O-(12-[L-leucinoylamino]dodecanoyl)8-O-debutanoylthapsigargin, also referred to herein as “L12ADT”. FIG. 7, for example, shows a chemical structure of thapsigargin analog modified in O-8 position with 12-aminododecanoyl side chain coupled to carboxyl-group of an amino acid.

In another preferred embodiment, the thapsigargin analog or derivative is 8-O-(12-aminododecanoyl)-8-O-debutanoyl thapsigargin (12ADT) (FIG. 8).

In some embodiments, for example with peptides susceptible to cleavage by PSA, hK2 and FAP, the peptide and therapeutic drug are linked directly or indirectly (by a linker) through the carboxy terminus of the peptide. See, for example, U.S. Pat. Nos. 7,906,477, 7,053,042, 6,265,540, 6,410,514, 6,504,014, 6,545,131, 7,635,682, and U.S. patent application Ser. Nos. 12/087,398 and 13/471,316.

In other embodiments, for example with peptides susceptible to cleavage by PSMA, the peptide and therapeutic drug are linked directly or indirectly (by a linker) to the α-amino terminus, the α-carboxy terminus, or a side chain of the peptide, as described in U.S. Pat. Nos. 7,767,648 and 7,468,354. In a further preferred embodiment in this embodiment, the linkage between the therapeutic drug and the peptide is a direct linkage occurring at either the amino terminus, or at the side chain of the peptide.

In both of the above embodiments, the site of attachment on the therapeutic drug must be such that, when coupled to the peptide, the non-specific toxicity of the drug is substantially inhibited. Thus the prodrugs should not be significantly toxic.

The active compounds of the invention may also comprise groups which enhance solubility of the active compound in the solvent in which the active compound is to be used. Most often the solvent is water, but may also include polysaccharides or other polyhydroxylated moieties. For example, dextan, cyclodextrin, starch and derivatives of such groups may be included in the peptide or prodrug of the invention. In a preferred embodiment, the group which provides solubility to the peptide or prodrug is a polymer, e.g., polylysine or polyethylene glycol (PEG).

Pharmaceutical Formulations

Pharmaceutical formulations for the compounds of the present invention are disclosed in, for example, U.S. patent application Ser. Nos. 12/087,398 and 13/257,131.

Pharmaceutical compositions comprising the active compounds of the invention are provided herein. These pharmaceutical compositions comprise the presently disclosed active compounds in a therapeutically effective amount in a pharmaceutically acceptable carrier.

Pharmaceutical formulations can be prepared for oral, intravenous, or aerosol administration as discussed in greater detail below. In a preferred embodiment, the presently disclosed subject matter provides active compounds that have been lyophilized and that can be reconstituted to form pharmaceutically acceptable formulations for administration, as by intravenous or intramuscular injection.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Useful injectable compositions include sterile suspensions, solutions or emulsions of the active compounds in aqueous or oily vehicles. In one embodiment, the compositions also can contain formulating agents, such as suspending, stabilizing and/or dispersing agents. The compositions suitable for injection can be presented in unit dosage form, e.g., in ampules or in multidose containers, and can contain added preservatives. Alternatively, an injectable composition can be provided in powder form for reconstitution with a suitable vehicle, including, but not limited to, sterile water, buffer, dextrose solution, and the like, before use. To this end, the active compounds of the invention can be dried by any art-known technique, such as lyophilization, and reconstituted prior to use.

For prolonged delivery, the active compounds can be formulated as a depot preparation for administration by implantation or intramuscular injection. The active compounds can be formulated with suitable polymeric or hydrophobic materials (e.g., as an emulsion in an acceptable oil) or ion exchange resins, or as sparingly soluble derivatives, e.g., as a sparingly soluble salt.

The pharmaceutical composition (or formulation) for application may be packaged in a variety of ways depending upon the method used for administering the drug. Generally, a kit or article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of active compounds of the invention may be prepared for various routes and types of administration. A compound having the desired degree of purity is optionally mixed with pharmaceutically acceptable diluents, carriers, excipients or stabilizers (Remington's Pharmaceutical Sciences (1980) 16th edition, Osol, A. Ed.), in the form of a lyophilized formulation, milled powder, or an aqueous solution. Formulation may be conducted by mixing at ambient temperature at the appropriate pH, and at the desired degree of purity, with physiologically acceptable carriers, e.g., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The active compounds for use herein are preferably sterile. The active compounds may be stored as a solid composition, a lyophilized formulation, or an aqueous solution. The pharmaceutical formulations described herein can be lyophilized using techniques well known in the art. In such embodiments, the active compound is provided in the form of a lyophilizate, which is capable of being reconstituted with a suitable pharmaceutically acceptable carrier to form a liquid formulation suitable for injection thereof into a subject.

In a preferred embodiment of the present invention, pharmaceutical formulations of the active compounds of the present invention include injectable emulsion compositions containing the active compounds of the present invention.

In one embodiment, the present invention provides the active compounds of the invention in a safe and commercially feasible injectable emulsion that is (a) free of any water-soluble and toxic solubilizer, such as organic solvents or surfactants, (b) sufficiently stable to provide an acceptable shelf life, (c) composed of small oil droplets and filterable though a 0.2-micron filter, (d) transparent or translucent and (e) lyophilizable.

In one embodiment, the emulsion of the present invention is an off-white, semi-transparent composition, which comprises oil droplets of an average size of less than about 200 nanometers in diameter, or more preferably, less than about 150 nm, or more preferably, less than about 75 nm. The emulsion is stable and has an excellent long-term stability in a lyophilized form. Chemically it maintains the integrity of the active compounds of the present invention and physically, it remains semi-transparent or translucent and maintains the nanometer droplet size upon prolonged storage (for example, at least 3 months, as shown in Examples 6 and 7). In addition, in some embodiments, the emulsion is characterized by high light transmittance and small droplet size even after three cycles of freeze-thaw treatment (see, e.g., Examples 2 and 4). Biologically, except for the active compound portion, the emulsion is non-allergenic, does not cause hypersensitivity or anaphylactic reactions, and is non-hemolytic. While not wishing to be bound by any specific theory, the disclosed injectable emulsions solubilize the lipophilic prodrugs of the present invention, presumably in the oil droplets.

In one embodiment, the present invention provides an injectable emulsion composition that contains a much lower oil concentration (about 2-3%) than what is disclosed in the prior art injectable emulsion (about 10-20%).

In another embodiment, the present invention provides an injectable emulsion composition that contains a much higher lecithin concentration (about 5-10%) than what is currently disclosed in the prior art injectable emulsion (less than about 2%).

In a preferred embodiment, the present invention provides an injectable emulsion composition that contains a much lower oil concentration (about 2-3%) than what is disclosed in the prior art injectable emulsion (about 10-20%) and contains a much higher lecithin concentration (about 5-10%) than what is currently disclosed in the prior art injectable emulsion (less than about 2%).

In a variation of these embodiments, the present invention provides an injectable emulsion composition that uses two oils consisting of an oil (for example, a soybean oil) and a medium chain triglyceride oil to form the oil phase, wherein the total oil concentration is up to 3%, and in a preferred embodiment is about 1-3%.

In yet another variation of these embodiments, the present invention provides an injectable emulsion composition that uses about 10-15% sucrose as the osmotic pressure modifier instead of the 2.2% glycerol used in the prior art injectable emulsions.

In a further variation of these embodiments, the present invention includes an emulsion composition comprising an active compound of the present invention at about 1.5% to about 2.5%, preferably at about 2%, an oil (for example, soybean oil) up to about 1.5%, preferably at about 0.5-1%, a medium chain triglyceride up to about 1.5%, preferably at about 0.5-1%, a lecithin at about 5-15%, preferably at about 5-10%, and sucrose at about 10-15%, all percentages based on the total weight of the composition.

In another variation of these embodiments, the lecithin-to-prodrug weight ratio is about 5:1 to about 7.5:1. In a further embodiment, the lecithin-to-oil weight ratio may be about 10:1 to about 5:1.

In yet another variation of these embodiments, the lecithin-to-prodrug weight ration is about 2.5:1 to about 5:1. In a variation of this embodiment, the lecithin-to-oil ratio may be about 10:1 to about 5:2.

The injectable emulsion compositions disclosed herein are capable of providing long-term shelf life for the active compounds of the present invention sufficient for a commercializable drug.

Being an oil-in-water emulsion, the injectable emulsion compositions of the invention differ significantly from the other prior art injectable emulsion compositions in their composition and properties. In one example, the traditional injectable emulsion for intravenous injection can be represented by the composition of Propofol Injectable Emulsion (DIPRIVAN®), which contains 10% vegetable oil as the oil phase, 1.8% egg lecithin as an emulsifier and 2.25% glycerol as the osmotic pressure modifier wherein the drug is dissolved in the oil droplets and the oil droplets are suspended in an aqueous phase. A similar composition is used to solubilize other insoluble drugs, such as in Clevidipine Injectable Emulsion (Cleviprex®), which contains 20% vegetable oil, 1.8% egg lecithin and 2.25% glycerol (see, for example, www.cleviprex.com/clev-pdfs/1%2014_Proposed_PI-S-003%20(clean %2Over).pdf)), and Diazepam Injectable Emulsion (DIAZEMULS®), which contains 15% vegetable oil, 1.2% egg lecithin and 2.2% glycerol (see, e.g., www.pfizer.ca/en/our_products/products/monograph/203). The general composition of these prior art injectable emulsions can be summarized as having about 10-20% vegetable oil, less than 2% lecithin and about 2.2% glycerol. Furthermore, these prior art injectable emulsions share similar physical properties, i.e. they are milky-white and opaque in appearance, have an average droplet size of about 300-400 nm in diameter, and are provided as aqueous liquid. None of these prior art emulsion compositions are available as dry powder, and therefore cannot be used for drugs that are sensitive to water such as the active compounds of the present invention.

In one embodiment, an injectable emulsion of the present invention is semi-transparent in appearance instead of being “milky-white and opaque.” Having the semi-transparent appearance or a high light transmittance permits the user to examine the emulsion for presence of any contamination or foreign matter before injection. This will help ensure the safety of the emulsion drug. An injectable emulsion of the present invention has a light transmittance of preferably at least about 30%, more preferably at least about 40%, even more preferably at least about 50%, and even more preferably at least about 60%, and even more preferably at least about 80% as compared to less than about 20% for a prior art emulsion such as Propofol Injectable Emulsion.

In another embodiment, an injectable emulsion of the present invention has a much smaller average droplet size than a prior art emulsion. The typical droplet size for an injectable emulsion for the compounds of the present invention is between 100 and 200 nm in diameter, and may be less than 100 nm in diameter. In contrast, the emulsion products disclosed in the prior art, such as Propofol Injectable Emulsion, have a typical droplet size between 300 and 400 nm. The smaller droplet size of the emulsions of the present invention permits sterilization of the emulsions by filtration through a 0.2-micron filter. Such filtration sterilization is important for an injectable composition of the prodrugs of the present invention since other sterilization methods such as heat, autoclave, radiation or gas can destroy the active compounds of the invention.

In one embodiment, the emulsion compositions of the invention have an average initial droplet size of no greater than about 200 nm in diameter, preferably no greater than about 175 nm in diameter, preferably no greater than about 150 nm in diameter, preferably no greater than 100 nm in diameter, and preferably no greater than 75 nm in diameter.

In another embodiment, the emulsion compositions of the invention maintain an average droplet size of no greater than about 200 nm in diameter, preferably no greater than about 175 nm in diameter, preferably no greater than about 150 nm in diameter, preferably no greater than 100 nm in diameter, and preferably no greater than 75 nm in diameter after freeze-thaw stress.

In another embodiment, the emulsion compositions of the present invention have an initial droplet size of no greater than about 200 nm in diameter and maintain an average droplet size of no greater than about 200 nm in diameter after freeze-thaw stress. Preferably, the emulsion compositions of the present invention have an initial droplet size of no greater than about 150 nm in diameter and maintain an average droplet size of no greater than about 200 nm in diameter after freeze-thaw stress.

In another variation of the embodiments of the invention disclosed herein, an injectable emulsion of the present invention can be lyophilized and provided in dry form. In contrast, the prior art emulsion cannot be lyophilized and can only be provided as an aqueous emulsion. The lyophilizable nature of the emulsion of the present invention provides long-term stability of the active compounds of the present invention and allows the emulsion of the present invention to be commercially feasible as a drug product. In one embodiment, the injectable emulsions of the present invention provide stability for the active compounds of the invention for at least 3 months, preferably at least 6 months, preferably at least 12 months, preferably at least 24 months, and, in some embodiments, are stable for at least 48 months in lyophilized form. Upon reconstitution, the emulsion compositions of the present invention will preferably maintain the components of the emulsion composition in their pre-lyophilized percentages. In some embodiments, the emulsion compositions are stable for about 6 hours to about 48 hours after reconstitution.

In one embodiment, the emulsion composition disclosed herein is an oil-in-water emulsion comprising an active compound of the present invention at about 1.5% to about 2.5%, preferably at about 2% of the total weight of the composition; a soybean oil or similar oil up to about 5%, preferably at about 0.5% to about 3%, and more preferably at about 0.5% to about 1% of the total weight of the composition; a medium chain triglyceride up to about 5%, preferably at about 0.5% to about 3%, and more preferably at about 0.5% to about 1% of the total weight of the composition, a lecithin at about 5% to about 15%, preferably at about 5% to about 12%, and more preferably at about 5% to about 10% of the total weight of the composition; and sucrose at about 8% to about 17%, preferably at about 10% to about 15% of the total weight of the composition.

In one embodiment, the emulsion composition disclosed herein is a dry (e.g., lyophilized) composition which, upon mixing with water, forms an oil-in-water emulsion comprising an active compound of the present invention at about 1.5% to about 2.5%, preferably at about 2% of the total weight of the composition; a soybean oil or similar oil up to about 5%, preferably at about 0.5% to about 3%, and more preferably at about 0.5% to about 1% of the total weight of the composition; a medium chain triglyceride up to about 5%, preferably at about 0.5% to about 3%, and more preferably at about 0.5% to about 1% of the total weight of the composition, a lecithin at about 5% to about 15%, preferably at about 5% to about 12%, and more preferably at about 5% to about 10% of the total weight of the composition; and sucrose at about 8% to about 17%, preferably at about 10% to about 15% of the total weight of the composition.

This invention further provides a process to prepare an oil-in-water emulsion comprising an active compound of the present invention at about 1.5% to about 2.5%, preferably at about 2% of the total weight of the composition; a soybean oil or similar oil up to about 5%, preferably at about 0.5% to about 3%, and more preferably at about 0.5% to about 1% of the total weight of the composition; a medium chain triglyceride up to about 5%, preferably at about 0.5% to about 3%, and more preferably at about 0.5% to about 1% of the total weight of the composition, a lecithin at about 5% to about 15%, preferably at about 5% to about 12%, and more preferably at about 5% to about 10% of the total weight of the composition; and sucrose at about 8% to about 17%, preferably at about 10% to about 15% of the total weight of the composition.

The invention further provides a process to prepare a dry (e.g., lyophilized) composition which upon mixing with water form an oil-in-water emulsion comprising an active compound of the present invention at about 1.5% to about 2.5%, preferably at about 2% of the total weight of the composition; a soybean oil or similar oil up to about 5%, preferably at about 0.5% to about 3%, and more preferably at about 0.5% to about 1% of the total weight of the composition; a medium chain triglyceride up to about 5%, preferably at about 0.5% to about 3%, and more preferably at about 0.5% to about 1% of the total weight of the composition, a lecithin at about 5% to about 15%, preferably at about 5% to about 12%, and more preferably at about 5% to about 10% of the total weight of the composition; and sucrose at about 8% to about 17%, preferably at about 10% to about 15% of the total weight of the composition (all percentages based on the total weight of the composition).

The emulsions of the present invention may further contain pharmaceutically acceptable additives including, but not limited to, acidifying agent, alkalizing agent, antioxidants, antimicrobial preservative, osmotic pressure modifying agents, cryo-protectants, and other injectable ingredients. In certain embodiments, such additives assist in stabilizing the emulsion and rendering sufficient shelf life to the compositions of the present invention. In preferred embodiments, the present compositions are both chemically and physically stable.

Dosages

The prodrugs of the invention, or compositions thereof, will generally be used in an amount effective to achieve the intended purpose. Of course, it is to be understood that the amount used will depend on the particular application.

For use to treat or prevent tumor or target cell growth or diseases related thereto, the prodrugs of the invention, or compositions thereof, are administered or applied in a therapeutically effective amount.

For systemic administration, a therapeutically effective dose can be estimated initially from in vitro assays. For example, a dose can be formulated in animal models to achieve a circulating prodrug concentration range that includes the IC₅₀ as determined in cell culture (e.g., the concentration of test compound that is lethal to 50% of a cell culture), the MIC, as determined in cell culture (e.g., the minimal inhibitory concentration for growth) or the IC₁₀₀ as determined in cell culture (e.g., the concentration of peptide that is lethal to 100% of a cell culture). Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

The amount of active compound administered will, of course, be dependent on the subject being treated, on the subject's weight, the severity of the affliction, the manner of administration and the judgment of the prescribing physician.

The therapy may be repeated intermittently. The therapy may be provided alone or in combination with other drugs, such as for example other antineoplastic entities or other pharmaceutically effective entities.

Toxicity

Preferably, a therapeutically effective dose of the active compounds described herein will provide therapeutic benefit without causing substantial toxicity.

Toxicity of the active compounds described herein can be determined by standard pharmaceutical procedures in cell cultures or experimental animals, e.g., by determining the LD₅₀ (the dose lethal to 50% of the population) or the LIMN (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the active compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingi et al., 1975, In: The Pharmacological Basis of Therapeutics, Ch.1, p.1).

Articles of Manufacture

In another embodiment of the invention, an article of manufacture, or “kit”, containing materials useful for the treatment of the disorders described above is provided. The article of manufacture comprises a container and a label or package insert on or associated with the container. Suitable containers include, for example, bottles, vials, syringes, blister pack, etc. The containers may be formed from a variety of materials such as glass or plastic. The container holds an active compound or formulation thereof (for example, a lyophilized, hydrated or rehydrated version of the injectable emulsions of the invention) effective for treating the condition and may have a sterile access port (for example the container may be an intravenous solution bag or a vial having a stopper pierceable by a hypodermic injection needle). At least one active agent in the composition is an active compound of the invention. The label or package insert indicates that the composition is used for treating the condition of choice, such as a cell proliferative disorder or cancer. In one embodiment, the label or package insert includes instructions for use and indicates that the composition comprising a compound of the invention and can be used to treat a cell proliferative disorder or cancer.

In another embodiment, the kit may comprise (a) a first container with an active compound of the invention or a formulation thereof contained therein; and (b) a second container with a second pharmaceutical formulation contained therein, wherein the second pharmaceutical formulation comprises a second compound with anti-tumor or anti-proliferative activity. The article of manufacture in this embodiment of the invention may further comprise a package insert indicating that the first and second compounds can be used to treat patients with a cell proliferative disorder, such as cancer. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Methods of Treating

The invention also provides methods of treating cell proliferative disorders or cancers that produce proteases that target the peptide sequences of the invention (for example, FAP, hK2, PSMA and PSA) with the active compounds of the invention. The active compounds of the invention and/or analogs or derivatives thereof can be administered to any host, including a human or non-human animal, in an amount effective to treat such a disorder.

As stated above, the active compounds of the invention can be administered parenterally by injection or by gradual infusion over time. The prodrugs can be administered intravenously, intraperitoneally, intramuscularly, subcutaneously, intracavity, or transdermally. Preferred methods for delivery of the active compounds of the invention include intravenous or subcutaneous administration. In one embodiment, a preferred method of delivery of the active compounds of the invention is via an injectable emulsion. Other methods of administration, as well as dosing regimens, will be known to those skilled in the art.

In one embodiment, the injectable emulsions described herein may be used to administer the active compounds of the invention for treating a cell proliferative disorder or cancer in a subject suspected of having cancer, a subject diagnosed with a cancer or a subject at risk for having cancer.

In one embodiment, the injectable emulsions described herein may be used to administer the active compounds of the invention for treating a cell proliferative disorder, including cancer, in a subject that is suspected of having, has been diagnosed with or is at risk for carcinoma, melanoma, lymphoma, blastoma, sarcoma, and leukemia or lymphoid malignancy. Further, in this embodiment, the injectable emulsions described herein may be used to administer the active compounds of the invention for treating cancer in a subject that has been diagnosed with or is at risk for epithelial cancers. In a further variation of this embodiment, the injectable emulsions described herein may be used to administer the active compounds of the invention for treating cancer in a subject that has been diagnosed with or is at risk for prostate, liver or breast cancer. In another embodiment, the injectable emulsions described herein may be used to administer the active compounds of the invention for treating a cell proliferative disorder, for example, BPH.

Combination Therapy Methods

Compounds of the invention may be combined in a pharmaceutical combination formulation, or dosing regimen as combination therapy, with a second compound that has anti-proliferative properties or that is useful for treating a cell proliferative disorder, including cancer. The second compound of the pharmaceutical combination formulation or dosing regimen preferably has complementary activities to the active compounds of the invention such that they do not adversely affect the other(s). Such molecules are suitably present in combination in amounts that are effective for the purpose intended.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities. Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, e.g. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, e.g. serially, whereas in combination therapy, effective dosages of two or more active ingredients are administered together.

As an example, the agent may be administered in combination with surgery to remove an abnormal proliferative cell mass. As used herein, “in combination with surgery” means that the agent may be administered prior to, during or after the surgical procedure. Surgical methods for treating epithelial tumor conditions include intra-abdominal surgeries such as right or left hemicolectomy, sigmoid, subtotal or total colectomy and gastrectomy, radical or partial mastectomy, prostatectomy and hysterectomy. In these embodiments, the agent may be administered either by continuous infusion or in a single bolus. Administration during or immediately after surgery may include a lavage, soak or perfusion of the tumor excision site with a pharmaceutical preparation of the agent in a pharmaceutically acceptable carrier. In some embodiments, the agent is administered at the time of surgery as well as following surgery in order to inhibit the formation and development of metastatic lesions. The administration of the agent may continue for several hours, several days, several weeks, or in some instances, several months following a surgical procedure to remove a tumor mass.

The subjects can also be administered the agent in combination with non-surgical anti-proliferative (e.g., anti-cancer) drug therapy. In one embodiment, the agent may be administered in combination with an anti-cancer compound such as a cytostatic compound. A cytostatic compound is a compound (e.g., a nucleic acid, a protein) that suppresses cell growth and/or proliferation. In some embodiments, the cytostatic compound is directed towards the malignant cells of a tumor. In yet other embodiments, the cytostatic compound is one that inhibits the growth and/or proliferation of vascular smooth muscle cells or fibroblasts.

Suitable anti-proliferative drugs or cytostatic compounds to be used in combination with the agents of the invention include anti-cancer drugs. Anti-cancer drugs are well known in the art and described, for example, in U.S. patent application Ser. No. 12/087,398.

According to the methods of the invention, the agents of the invention may be administered prior to, concurrent with, or following the other anti-cancer compounds. The administration schedule may involve administering the different agents in an alternating fashion. In other embodiments, the agent may be delivered before and during, or during and after, or before and after treatment with other therapies. In some cases, the agent is administered more than 24 hours before the administration of the other anti-proliferative treatment. In other embodiments, more than one anti-proliferative therapy may be administered to a subject. For example, the subject may receive the agents of the invention, in combination with both surgery and at least one other anti-proliferative compound. Alternatively, the agent may be administered in combination with more than one anti-cancer drug.

Methods of Making Prodrug Compounds

The invention provides a method of producing the active compounds of the invention. This method involves linking a therapeutically active drug to a peptide of the invention described above. In certain embodiments the peptide is linked directly to the drug; in other embodiments the peptide is indirectly linked to the drug via a linker. In certain embodiments, the carboxy terminus of the peptide is used for linking, for example, with prodrugs susceptible to cleavage by PSA, hK2 and FAP. In other embodiments, the amino terminus of the peptide is used for linking, for example, with prodrugs susceptible to cleavage by PSMA. One example of making a PSMA prodrug of the present invention is disclosed in U.S. Application No. 61/791,909.

In some embodiments, the therapeutic drug contains a primary amine to facilitate the formation of an amide bond with the peptide. Many acceptable methods for coupling carboxyl and amino groups to form amide bonds are known to those skilled in the art.

The peptide may be coupled to the therapeutic drug via a linker. Suitable linkers include, but are not limited to, any chemical group that contains a primary amine and include amino acids, primary amine-containing alkyl, alkenyl or arenyl groups. The connection between the linker and the therapeutic drug may be of any type know in the art, preferably covalent bonding.

Linkers that may be used with the peptide prodrugs of the present invention have also been described in U.S. Pat. Nos. 7,906,477; 7,053,042; 7,767,648; 7,468,354; 6,265,540; 6,504,014; 6,410,514; 6,545,131; and 7,635,682, and U.S. patent application Ser. Nos. 12/087,398, 13/471,316 and 13/257,131.

In certain embodiments, the linker comprises an amino acid or amino acid sequence. The sequence may be of any length, but is preferably between 1 and 10 amino acids, most preferably between 1 and 5 amino acids.

The prodrug compounds can be prepared according to standard synthetic or recombinant techniques known to those of skill in the art. For instance, peptide linking moieties can be synthesized by conventional solid phase or solution phase peptide chemistry. Biologically active entities and masking moieties can be obtained from commercial sources or from other well-known methods such as purification from natural sources, recombinant expression and other techniques. Dual polarity linkers and spacer moieties can be synthesized or obtained from commercial sources or from other well-known methods.

Typically, the prodrugs are prepared synthetically by condensing the masking moiety and biologically active entity with the linking moiety. Well known protecting groups can be used advantageously in the preparation of prodrug compounds. If the linking moiety is a peptide and the biologically active entity is a polypeptide and a terminus of the linking moiety is linked to a complementary terminus of the biologically active entity via an amide bond, the prodrug, or a portion thereof, can conveniently be prepared by recombinant synthesis. A nucleic acid coding for the amino acid sequence of the linking moiety and the biologically active agent can be prepared and used to express the covalent linking moiety-biologically active agent complex by standard techniques (see, e.g., Ausubel et al., 1987, Current Protocols in Molecular Biology, John Wiley & Sons, Inc., New York). The masking moiety can then be linked, for instance, to the amino terminus of the linking moiety by standard solution phase peptide chemistry. If the masking moiety is also a peptide or polypeptide and a terminus of the masking moiety is also linked to a complementary terminus of the linking moiety via an amide bond, the entire prodrug can conveniently be prepared by recombinant synthetic techniques. The nucleic acid expressing the prodrug should encode the amino acid sequences of the masking moiety, the linking moiety and the biologically active entity in tandem. Prodrugs produced by recombinant synthesis can be expressed in any eukaryotic or prokaryotic system in which the linking moiety is not cleaved by proteases, peptidases or other factors.

The invention will now be described in greater detail by reference to the following non-limiting examples.

### Example 1

### Preparation of An Emulsion containing Ser-Ser-Lys-Tyr-Gln-L12ADT (SEQ ID NO: 18)

An emulsion containing the thapsigargin derivative 8-O-(12-[L-leucinoylamino]dodecanoyl)-8-O-debutanoylthapsigargin (L12ADT) linked to the carboxy terminus of the peptide Ser-Ser-Lys-Tyr-Gln (SEQ ID NO: 18) (referred to herein as “G-115”) was prepared using the following composition and procedure:

G-115 F9 Composition

| Ingredient | Grade | Supplier | % (w/w) |
|---|---|---|---|
| G-115 | N/A | Cedarburg Hauser | 2 |
| | | Pharmaceuticals | |
| Soybean oil, super refined | USP | Croda | 0.5 |
| Medium chain triglyceride | DMF | Sasol Inc. | 0.5 |
| (Miglyol 812 ®) | | | |
| Soy lecithin, LIPOID S-100 ® | EP | Lipoid | 10 |
| Sucrose | NF | Spectrum | 15 |
| | | Chemicals | |
| Water-for-injection (WFI) | USP | Hospira, Inc. | qs to 100 |
| NaOH/HCl to adjust pH | | | |

### Procedure

1. Weigh out and transfer soybean oil, medium chain triglyceride, soy lecithin, and sucrose, along with about 80% batch size of deionized water, into a container of a suitable size. Record weight of each component added.
2. Homogenize using a high shear mixer (e.g., Silverson Model L5) at about 5000 RPM for about 2 minutes to obtain a crude emulsion.
3. Weigh out and transfer batch amount of G-115 into the emulsion. High shear to dissolve all solids.
4. Homogenize and adjust pH to between 3-4 using 1N hydrochloric acid or sodium hydroxide.
5. Add deionized water to total batch weight.
6. Homogenize using a microfluidizer (e.g., Model M110EH, Microfluidics Corporation), until the average droplet size, as determined by laser light scattering (LLS) method (e.g., Model NanoZS, Malvern Zeta sizer), is less than 150 nm.
7. Filter the emulsion through a 0.22 μm filter to sterilize the emulsion.
8. Aseptically, fill the filtered emulsion into sterile vials.
9. Crimp seal the vials.

The G-115 F9 emulsion thus prepared is off-white and semi-transparent solution with droplet size less than 150 nm.

### Example 2

### Compositions and Droplet Sizes of Emulsions Containing G-115

Various emulsions containing 2% w/w G-115 were prepared using the following compositions by the procedure described in Example 1:

| Ingredient (% w/w) | F1 | F2 | F3 | F4 | F5 | F6 | F7 | F8 | F9 | F10 | F11 | F12 |
|---|---|---|---|---|---|---|---|---|---|---|---|---|
| G-115 | 2 | 2 | 2 | 2 | 2 | 2 | 2 | 2 | 2 | 2 | 2 | 2 |
| Soy bean oil | | | | | 0.5 | 1 | 3 | 5 | 0.5 | 1 | 3 | 5 |
| Medium chain | | | | | 0.5 | 1 | 3 | 5 | 0.5 | 1 | 3 | 5 |
| triglycide | | | | | | | | | | | | |
| Soy lecithin | 5 | 10 | 12.5 | 15 | 5 | 5 | 5 | 5 | 10 | 10 | 10 | 10 |
| Sucrose | 10 | 10 | 10 | 10 | 15 | 15 | 15 | 15 | 15 | 15 | 15 | 15 |
| Deionized | 100 | 100 | 100 | 100 | 100 | 100 | 100 | 100 | 100 | 100 | 100 | 100 |
| Water QS to | | | | | | | | | | | | |
| Initial droplet | 99.3 | 116.7 | 133 | 132.7 | 69.6 | 89.9 | 114.7 | 146.0 | 76.8 | 88.2 | 161.7 | 180.3 |
| size (nm) | | | | | | | | | | | | |
| Droplet size | 343.7 | 123.7 | 127.3 | 131.0 | 202.3 | 369.7 | 376.7 | 370.0 | 84.2 | 103.7 | 166.0 | 189.7 |
| after 3 freeze- | | | | | | | | | | | | |
| thaw cycles (nm) | | | | | | | | | | | | |

The average droplet size was determined for each composition by laser light scattering (LLS) method (e.g., Model NanoZS, Malvern Zeta sizer).

Compositions that exhibited an initial droplet size of less than 150 nm and a droplet size of less than 200 nm after 3 freeze-thaw cycles were observed in F2, F3, F4, F9 and F10. These compositions included G-115 at about 2%, soybean oil at about 0-1%, medium chain triglyceride at about 0-1%, a lecithin at about 10-15% and sucrose at about 10-15%. The lecithin-to-drug weight ratio in these compositions was determined to be about 5:1 to 7.5:1. For the compositions of F9 and F10, the lecithin-to-oil ratio was determined to be about 10:1 to 5:1. It was also observed that compositions F9 and F10, which included oil in the composition, exhibited smaller initial droplet sizes and smaller droplet sizes after three freeze-thaw cycles than F2, F3, and F4, which did not contain oil.

### Example 3

### Preparation of an Emulsion Containing the Thapsigargin Derivative 8-O-(12-aminododecanoyl)-8-O-debutanoyl thapsigargin (12ADT) Linked to the Aspartic Acid of a Peptide Having the Sequence Asp-Glu*Glu*Glu*Glu (SEQ ID NO: 486) (Referred to Herein as “G-202”)

An emulsion containing 2% G-202 was prepared using the following composition and similar procedure as described in Example 1:

G-202 F9 Composition

| Ingredient | Grade | Supplier | % (w/w)* |
|---|---|---|---|
| G-202 | N/A | Hauser | 2** |
| | | Pharmaceuticals | |
| Soybean oil, Super-refined | USP | Croda | 0.5 |
| Medium chain triglyceride | USP/EP | Sasol Inc. | 0.5 |
| (Miglyol 812 ®) | | | |
| Soy lecithin, LIPOID S- | EP | Lipoid | 10 |
| 100 ® | | | |
| Sucrose | NF | Spectrum Chemicals | 15 |
| Water-for-injection (WFI) | USP | Hospira Inc. | qs to 100 |
| Arginine | USP | Spectrum Chemicals | To adjust |
| | | | pH to |
| | | | 6.0 +/− 0.2 |

*Measured density is 1.026 g/mL at room temperature
**Equivalent to 2.052 mg/mL

### Procedure:

1. Weigh out and transfer batch amount of lecithin, soybean oil, medium chain triglycerides, sucrose and about 80% batch amount of WFI into a container of a suitable size. Record weight of each component added.
2. Homogenize using a high shear mixer (e.g. Silverson Model L5) at about 5000 RPM for about 2 minutes to form a primary emulsion.
3. Weigh out and transfer batch amount of G-202 into the emulsion. High shear to dissolve all solids.
4. Rinse the mixer head with 1-2% batch size of WFI. Collect the rinse and transfer back to the primary emulsion.
5. Adjust pH of the crude emulsion to 6.5+/−0.2 with a 1N arginine solution.
6. Add more WFI to the target batch size. High shear to form a uniform primary emulsion.
7. Using a microfluidizer (Model M110EH, Microfluidics Corporation), microfluidize the primary emulsion for 10 passes to reduce the droplet size to less than 150 nm in average diameter as determined by a laser light scattering (LLS) method (Model NanoZS, Malvern Zeta sizer).
8. Measure the pH. Adjust the pH to 6.0+/−0.2 with 1N arginine as needed.
9. In a biosafety hood, aseptically filter the microfluidized emulsion through a sterile 0.2 μm filter (Polyethersulfone, Nalgene) into a sterile receiving container.
10. Fill the filtered emulsion into sterile USP Type I 2-mL glass vials (supplier: Schott, cat#:68000314) and crimp seal with sterile rubber closure (supplier: Fisher Scientific, cat#:06447D).

The G-202 F9 emulsion thus prepared is off-white and semi-transparent solution with droplet size less than 150 nm.

### Example 4

### Compositions and Droplet Sizes of Emulsions Containing G-202

Emulsions containing 2% G-202 were prepared using the following compositions by the procedure described in Example 3:

| Ingredient (%, w/w) | F9 | F101 | F102 | F103 | F104 | F105 | F106 |
|---|---|---|---|---|---|---|---|
| G-202 | 2 | 2 | 2 | 2 | 2 | 2 | 2 |
| Soybean oil | 0.5 | 1 | 3 | 0.5 | 1 | 0.5 | 1 |
| Medium chain | 0.5 | 1 | 3 | 0.5 | 1 | 0.5 | 1 |
| triglyceride | | | | | | | |
| Soy lecithin | 10 | 10 | 10 | 12 | 12 | 5 | 5 |
| Sucrose | 15 | 10 | 10 | 10 | 10 | 10 | 10 |
| Water QS to | 100 | 100 | 100 | 100 | 100 | 100 | 100 |
| Initial droplet | 112.3 | 191.2 | 199.2 | 172.0 | 183.7 | 136.4 | 140.6 |
| size (nm) | | | | | | | |
| Droplet size | 113.5 | 201.1 | 205.0 | 166.3 | 221.8 | 139.3 | 140.1 |
| after 3 freeze- | | | | | | | |
| thaw cycles (nm) | | | | | | | |

The average droplet size was determined for each composition by laser light scattering (LLS) method (e.g., Model NanoZS, Malvern Zeta sizer).

Compositions that exhibited an initial droplet size of less than 150 nm and a droplet size of less than 200 nm after 3 freeze-thaw cycles were observed in F9, F105, and F106. These compositions contained G-202 at about 2%, soybean oil at about 0.5-1%, medium chain triglyceride at about 0.5-1%, a lecithin at about 5-10% and sucrose at about 10-15%. The lecithin-to-drug weight ratio is about 2.5:1 to 5:1, and the lecithin-to-oil ratio is about 10:1 to 5:2.

### Example 5

### Lyophilization

The F9 compositions comprising G-115 (Example 1) and G-202 (Example 3) were further lyophilized using the process described below:
1. Fill 1.0 mL of the filtered emulsion into a 2-mL USP type I glass vial (Schott, cat#68000314). Place stopper half-way down on the vial (13 mm gray butyl stopper, Wheaton, cat#06447E)
2. Place the vials in a lyophilizer (FTS System Model:Dura Stop μP) with the following freeze-dry cycle conditions:

### Freezing Phase Parameters:

| Step # | Ramp at (° C./min) | Temp (° C.) | Hold Time (min) |
|---|---|---|---|
| 1 | 2.5 | −30 | 180 |

### Drying Phase Parameters:

| | Ramp at | | Hold Time | Vacuum |
|---|---|---|---|---|
| Step | (° C./min) | Temp (° C.) | (min) | (mTorr) |
| A | 2.5 | −30 | 30 | 60 |
| B | 2.5 | −30 | 2400 | 60 |
| C | 2.5 | 4 | 900 | 60 |
| D | 2.5 | 25 | 240 | 60 |

3. After lyophilization, back-fill the vials with filtered nitrogen gas, NF and seal the vials at about 95% atmospheric pressure.
4. For reconstitution, add 0.75 mL WFI into each vial. Invert the vial repeatedly by hand for at least 2 minutes or until all solid mass is dissolved to form a uniform emulsion.

This lyophilization method produced off-white, uniform cake-like dry form in the vials for each formulation. Up on addition to water to the vial containing a lyophile to the pre-lyophilization weight, the lyophile formed off-white and semi-transparent solution. Droplet size of the reconstituted emulsion was determined for each composition as shown in Examples 2 and 4.

### Example 6

### Stability of an Emulsion Containing G-115

A lot of G-115 F9 lyophilized emulsion (lot#141-1-46) was prepared according to Examples 1 and 5 and were tested for stability. The stability tests included appearance, pH, G-115 assay and purity by HPLC, and average droplet size as determined by laser light scattering.

### Appearance of the Lyophilized Cake

| Sample | Initial (Time-0) | 3.5 months |
|---|---|---|
| −20° C. | Off white, uniform cake-like solid | Same as T-0 |
| 2-8° C. | | Same as T-0 |
| 25° C. | | Same as T-0 |

### Appearance of the Reconstituted Emulsion

| Sample | Initial (Time-0) | 3.5 months |
|---|---|---|
| −20° C. | Off white, semi-transparent solution. No | Same as T-0 |
| 2-8° C. | visible oil droplets or solid particles. | Same as T-0 |
| 25° C. | | Same as T-0 |

### Optical Microscopic Observation of the Reconstituted Emulsion (200× Magnification)

| Sample | Initial (Time-0) | 3.5 months |
|---|---|---|
| −20° C. | Clear, no solid particles or crystals | Same as T-0 |
| 2-8° C. | | Same as T-0 |
| 25° C. | | Same as T-0 |

pH

| Sample | Initial (Time-0) | 3.5 months |
|---|---|---|
| −20° C. | 3.29 | 3.56 |
| 2-8° C. | | 3.64 |
| 25° C. | | 3.68 |

G-115 Assay (mg/vial)

| Sample | Initial (Time-0) | 3.5 months |
|---|---|---|
| −20° C. | 10.8 | 10.9 |
| 2-8° C. | | 10.8 |
| 25° C. | | 10.0 |

% Label claim (Label claim=20 mg/vial)

| Sample | Initial (Time-0) | 3.5 months |
|---|---|---|
| −20° C. | 107.8 | 108.5 |
| 2-8° C. | | 108.3 |
| 25° C. | | 100.4 |

### Chromatographic Purity % (% Peak Area)

| Sample | Initial (Time-0) | 3.5 months |
|---|---|---|
| −20° C. | 98.7 | 98.7 |
| 2-8° C. | | 98.1 |
| 25° C. | | 98.9 |

### Average Droplet Size by Laser Light Scattering Method (Nm)

| Sample | Initial (Time-0) | 3.5 months |
|---|---|---|
| −20° C. | 138.0 | 150.1 |
| 2-8° C. | | 137.9 |
| 25° C. | | 145.5 |

Conclusion: G-115 F9 in lyophilized form is physically and chemically stable at −20, 2-8 and 25° C. for 3.5 months.

### Example 7

### Stability of an Emulsion Containing G-202

A lot of G-202 F9 lyophilized emulsion (lot#141-2-1) was prepared according to Examples 3 and 5 and tested for stability. The stability tests included appearance, pH, G-202 assay and purity by HPLC, and average droplet size as determined by laser light scattering.

### Appearance of Lyophilized Cake

| Sample | Initial (Time-0) | 1 month | 3 months |
|---|---|---|---|
| −20° C. | Off white, solid cake | Same as T-0 | Same as T-0 |
| 2-8° C. | | Same as T-0 | Same as T-0 |
| 25° C. | | Same as T-0 | Same as T-0 |

### Appearance of the Reconstituted Emulsion

| Sample | Initial (Time-0) | 1 month | 3 months |
|---|---|---|---|
| −20° C. | Semi-translucent, off white, | Same as T-0 | Same as T-0 |
| 2-8° C. | uniform solution. No visible oil | Same as T-0 | Same as T-0 |
| 25° C. | droplets or solid particles. | Same as T-0 | Same as T-0 |

### Microscopic Observation (200× Magnification)

| Sample | Initial (Time-0) | 1 month | 3 months |
|---|---|---|---|
| −20° C. | Clear, no solid particles or | Same as T-0 | Same as T-0 |
| 2-8° C. | crystals | Same as T-0 | Same as T-0 |
| 25° C. | | Same as T-0 | Same as T-0 |

pH

| Sample | Initial (Time-0) | 1 month | 3 months |
|---|---|---|---|
| −20° C. | 6.14 | 6.30 | 6.32 |
| 2-8° C. | | 6.25 | 6.26 |
| 25° C. | | 6.24 | 6.25 |

G-202 Assay (mg/vial)

| Sample | Initial (Time-0) | 1 month | 3 months |
|---|---|---|---|
| −20° C. | 21.5 | 20.9 | 20.7 |
| 2-8° C. | | 20.7 | 20.7 |
| 25° C. | | 19.9 | 20.0 |

% Label Claim (Label Claim=20 mg/Vial)

| Sample | Initial (Time-0) | 1 month | 3 months |
|---|---|---|---|
| −20° C. | 107.7 | 104.7 | 103.5 |
| 2-8° C. | | 103.7 | 103.7 |
| 25° C. | | 99.5 | 100.2 |

### Chromatographic Purity % (% Peak Area)

| Sample | Initial (Time-0) | 1 month | 3 months |
|---|---|---|---|
| −20° C. | 97.1 | 97.4 | 96.4 |
| 2-8° C. | | 97.7 | 96.0 |
| 25° C. | | 95.7 | 97.0 |

### Average Droplet Size by Laser Light Scattering Method (Nm)

| Sample | Initial (Time-0) | 1 month | 3 months |
|---|---|---|---|
| −20° C. | 65.2 | 61.8 | 59.7 |
| 2-8° C. | | 63.2 | 82.7 |
| 25° C. | | 63.6 | 65.2 |

Conclusion: G-202 F9 in lyophilized form is physically and chemically stable at −20, 2-8 and 25° C. for 3 months.

### Example 8

### Determination of Light Transmittance Value

A lot of G-202 F9 lyophilized emulsion (lot#141-2-1L) was reconstituted with water-for-injection to about 20 mg/mL. The reconstituted emulsion was placed in a 1-mm cell for light transmittance measurement (Model: Pharmacia LKB, Ultraspec III). The light transmittance measured at 600 nm was 91.6%.

### Example 9

### Comparison of Pharmacokinetic Profiles

The objective of this study was to assess and compare the pharmacokinetic profiles of G-202 formulated with polypropylene glycol/polysorbate 20 (Formulation A) versus G-202 formulated as a nanoemulsion (G-202 F9, Formulation B) following a single-dose intravenous slow bolus injection to male cynomolgus monkeys. The diluents were polyproplyene glycol, polysorbate 20, and 0.9% sodium chloride for Formulation A and water for injection and 5% dextrose for Formulation B. This study was a crossover design using two different formulations of the test article and two dosing events. Animals were fasted overnight and fed approximately 2-hours postdose. There was an approximate 3-week washout period between dose events. The monkeys were non-naïve with respect to prior drug treatment but were considered to be in good health based on physical examinations and clinical pathology evaluations completed prior initiation of the study. The animals were dosed according to the experimental design shown in the table below. Two different formulations of the test article were used in each of two dosing sessions (events). Animals were fasted overnight and fed approximately 2 hours postdose. There was an approximate 3-week washout period between dose events on Days 1 and 20.

| | | | G-202 | Dose | | |
|---|---|---|---|---|---|---|
| | Dose | | Dose | Concen- | Dose | Number |
| Dose | Event/ | Formu- | Level | tration | Volume | of |
| Group | Day | lation | (mg/kg) | (mg/mL) | (mL/kg) | Animals |
| 1 | 1/1 | A | 1 | 0.4 | 2.5 | 2 |
| 2 | 1/1 | B | 1 | 0.4 | 2.5 | 2 |
| 1 | 2/20 | B | 1 | 0.4 | 2.5 | 2 |
| 2 | 2/20 | A | 1 | 0.4 | 2.5 | 2 |

On each day of dosing, blood samples (approximately 1 mL/sample) were taken from each monkey at the time points listed below. All samples were collected within the specified windows.

Sample Collection Schedule

| | |
|---|---|
| Time Point | Window |

| Pre | NA | |
|---|---|---|
| 0.25 h (15 min) | ±1 | min |
| 0.5 h (30 min) | ±2 | min |

| | | | |
|---|---|---|---|
| 1 | h | ±5 | min |
| 3 | h | ±5 | min |
| 9 | h | ±20 | min |
| 12 | h | ±20 | min |
| 24 | h | ±20 | min |
| 48 | h | ±20 | min |

Concentrations were determined for each formulation after each dose event. In addition, samples of each vehicle preparation used to prepare Formulations A and B were collected. Dosing solutions for Formulation B met acceptable criteria (96 and 95% of the nominal 0.4 mg/mL drug concentration). For Formulation A, analyses of the drug concentration was lower (77 and 79% of the nominal 0.4 mg/mL) than acceptable criteria (±10%); therefore, the actual concentrations were used and the dose normalized values were used for the comparison of selected pharmacokinetic values.

Blood samples (approximately 1 mL/sample) were collected via the femoral vein from each monkey prior to dosing and at 0.25, 0.5, 1, 3, 9, 12, 24, and 48 hours postdose. The plasma was stored frozen until analysis via an LC-MS/MS method.

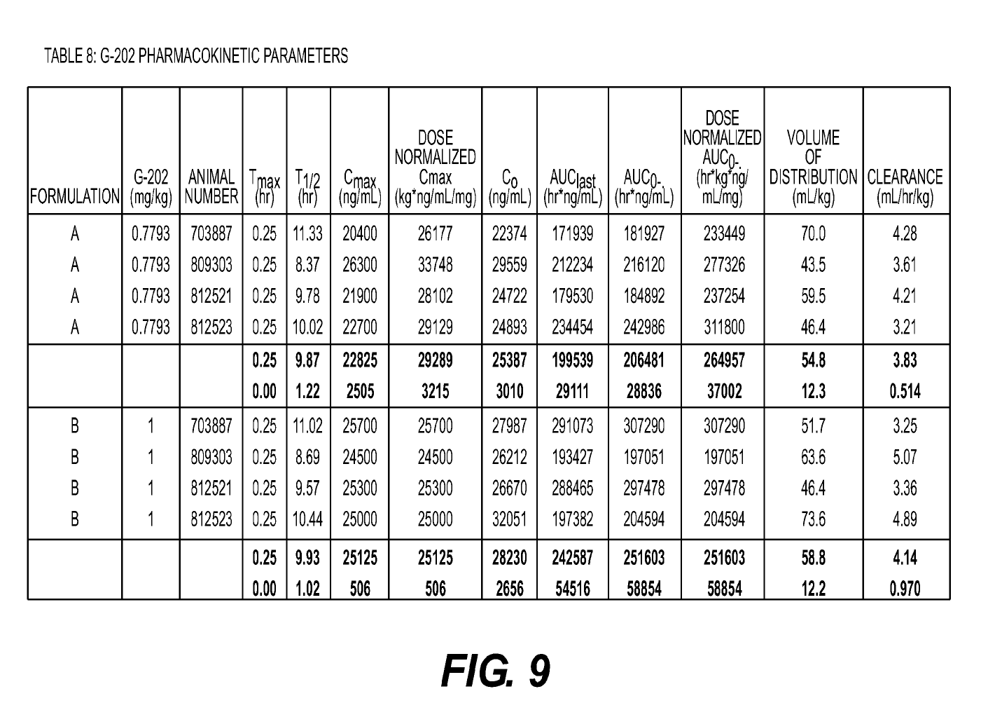

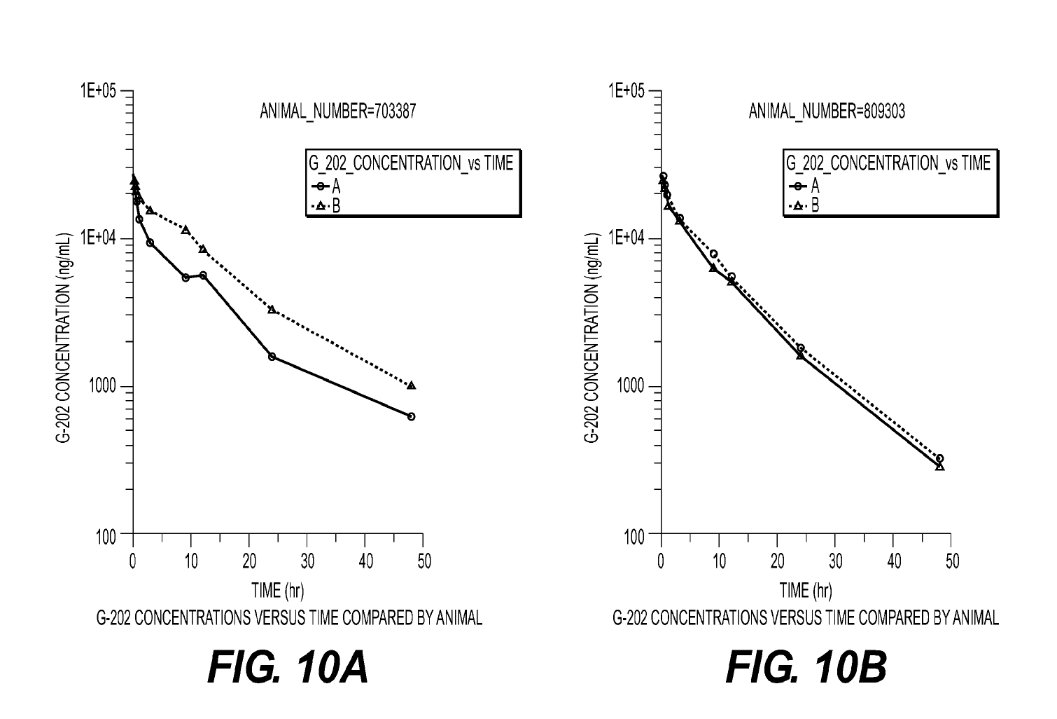

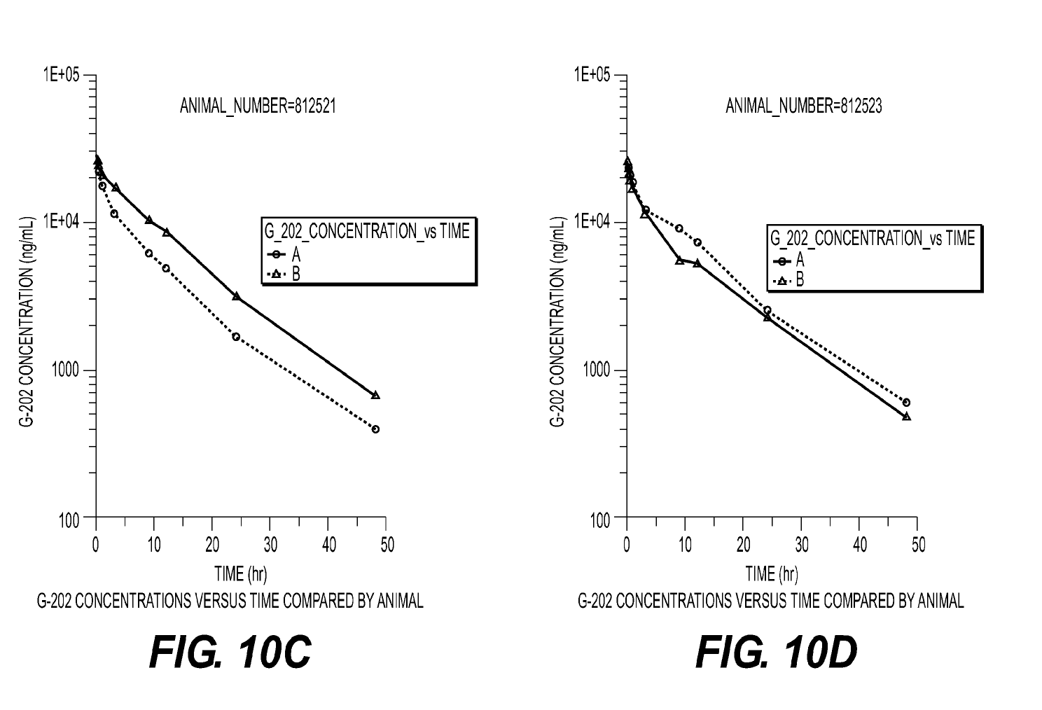

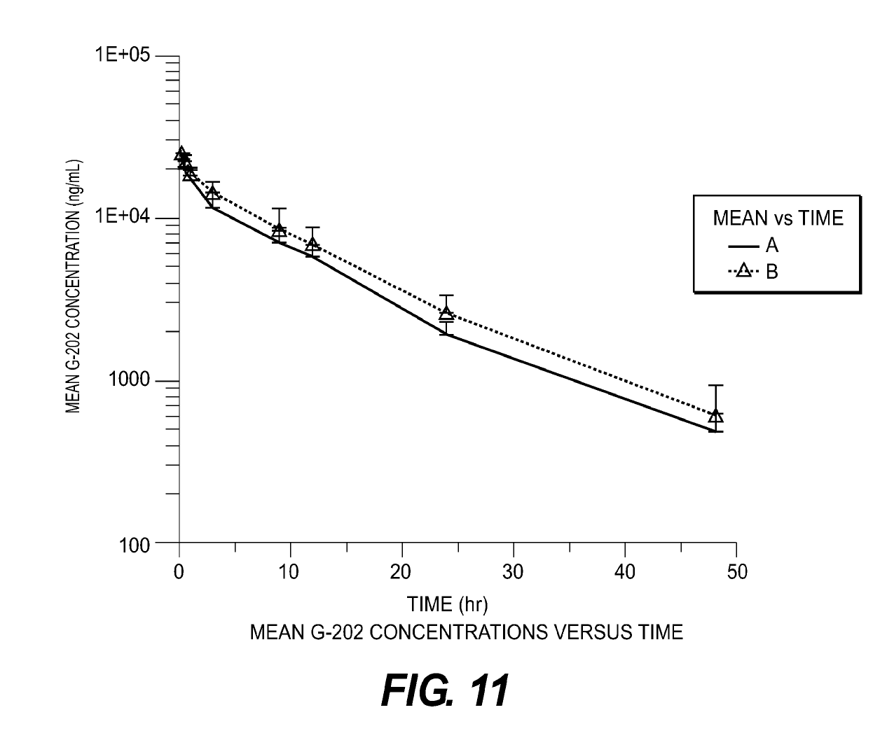

Pharmacokinetic parameters for G-202 in the two formulations are shown in Table 5 (FIG. 9). Graphs for individual animal G-202 concentration versus time are shown in FIG. 10. Graphs of mean G-202 concentration versus time are shown in FIG. 11.

Mean (±SD) pharmacokinetic parameters for the two formulations are shown in the table below.

| | | | | Dose Nor- | Dose Nor- | Volume of | |
|---|---|---|---|---|---|---|---|
| Formu- | G-202 | Tₘₐₓ | T_{1/2} | malized Cₘₐₓ | malized AUC₀₋_{_{—}} | Distribution | Clearance |
| lation | (mg/kg) | (hr) | (hr) | (kg*ng/mL/mg | (hr*kg*ng/mL/mg) | (mL/kg) | (mL/hr/kg) |
| A | 0.7793 | 0.25 ± 0.00 | 9.87 ± 1.22 | 29289 ± 3215 | 264957 ± 37002 | 54.8 ± 12.3 | 3.83 ± 0.514 |
| B | 1 | 0.25 ± 0.00 | 9.93 ± 1.02 | 25125 ± 506 | 251603 ± 58854 | 58.8 ± 12.2 | 4.14 ± 0.970 |

For both formulations, the mean Tₘₐₓ was observed at 0.25 hours, the first sampling point postdose. The G-202 elimination half-life was similar between the formulations; the mean half-life for Formulation A was 9.87 hours, and the mean half-life for Formulation B was 9.93 hours. The volume of distribution was also similar between the two formulations (54.8 mL/kg for Formulation A, 58.8 mL/kg for Formulation B). Clearance values were similar between the two formulations (3.83 mL/hr/kg for Formulation A, 4.14 mL/hr/kg for Formulation B).

Because of the differences in dose levels between the two formulations, Cₘₐₓ and AUC_{0-∞} were compared on a dose-normalized basis. The mean dose-normalized AUC_{0-∞} values were similar between the two formulations (265,000 hr*kg*ng/mL/mg for Formulation A, 252,000 hr*kg*ng/mL/mg for Formulation B). The mean dose-normalized Cₘₐₓ values were also similar between the two formulations albeit slightly higher for Formulation A (29,300 kg*ng/mL/mg for Formulation A, 25,100 kg*ng/mL/mg for Formulation B).

Based on the data from this study, the pharmacokinetics of the two formulations are similar.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps of the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

Other embodiments are set forth within the following claims.

What is claimed is:

1. A pharmaceutical composition suitable for intravenous administration comprising:
(a) a prodrug comprising
a therapeutically active drug comprising a sesquiterpene-γ-lactone, analogue or derivative thereof, and
a peptide comprising an amino acid sequence having a cleavage site specific for a protease associated with a cell proliferative disorder,
wherein the peptide has 20 or fewer amino acids in length,
wherein the peptide is linked to the therapeutically active drug to inhibit the therapeutic activity of the drug, and wherein the therapeutically active drug is cleaved from the peptide upon proteolysis by said protease; and
(b) a pharmaceutically acceptable vehicle;
wherein the prodrug is present in an amount of from about 1.5% to about 2.5% by weight of the total composition.

2. The composition of claim 1, wherein said pharmaceutically acceptable vehicle comprises
a lecithin or phospholipid in an amount of from about 5% to about 15% by weight of the total composition, and
a sucrose in an amount of from about 8% to about 17% by weight of the total composition.

3. The composition of claim 2, wherein said pharmaceutically acceptable vehicle further comprises
an oil in an amount up to about 5% by weight of the total composition, and
a medium chain triglyceride in an amount up to about 5% by weight of the total composition.

4. The composition of any one of claim 3, wherein said pharmaceutically acceptable vehicle comprises
oil in an amount of from about 0.5% to about 3% by weight of the total composition,
a medium chain triglyceride in an amount of from about 0.5% to about 3% by weight of the total composition,
a lecithin or phospholipid in an amount of from about 5% to about 12% by weight of the total composition, and
sucrose in an amount of from about 10% to about 15% by weight of the total composition.

5. (canceled)

6. The composition of claim 5, wherein said prodrug is present in an amount of about 2% by weight of the total composition.

7. (canceled)

8. (canceled)

9. (canceled)

10. (canceled)

11. (canceled)

12. (canceled)

13. The composition of claim 2, wherein the lecithin-to-prodrug weight ratio is about 2.5:1 to 5:1.

14. The composition of claim 3, wherein the lecithin-to-prodrug weight ratio is about 2.5:1 to 5:1.

15. The composition of claim 3, wherein the lecithin-to-oil weight ratio is about 10:1 to 5:2.

16. (canceled)

17. The composition of claim 13, wherein said prodrug comprises the thapsigargin derivative 8-O-(12-aminododecanoyl)-8-O-debutanoyl thapsigargin (12ADT) linked to the aspartic acid of a peptide having the sequence Asp-Glu*Glu*Glu*Glu (SEQ ID NO: 486), wherein at least one of the bonds designated with * is a gamma carboxy linkage.

18. (canceled)

19. The composition of claim 2, wherein said composition may be filtered through a 0.2-micron filter and/or may be filtered through a 0.2-micron filter after a freeze-thaw stress.

20. The composition of claim 3, wherein said composition may be filtered through a 0.2-micron filter and/or may be filtered through a 0.2-micron filter after a freeze-thaw stress.

21. (canceled)

22. (canceled)

23. (canceled)

24. (canceled)

25. (canceled)

26. (canceled)

27. (canceled)

28. (canceled)

29. (canceled)

30. (canceled)

31. A composition according to claim 2 wherein the oil droplets of said composition have an average diameter of less than about 200 nanometers or have an average diameter of less than about 200 nanometers after a freeze-thaw stress.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A composition according to claim 2 that exhibits a light transmission value at 600 nm of no less than about 30%.

47. A composition according to claim 3 that exhibits a light transmittance value at 600 nm of no less than about 30%.

48. (canceled)

49. (canceled)

50. (canceled)

51. (canceled)

52. A composition according to claim 51 that exhibits a light transmittance value at 600 nm of no less than about 60%.

53. The composition of claim 2 wherein said composition is lyophilizable and/or chemically stable for at least 3 months.

54. The composition of claim 3 wherein said composition is lyophilizable and/or chemically stable for at least 3 months.

55. (canceled)

56. The composition of claim 2, wherein said sesquiterpene-γ-lactone is a thapsigargin or a thapsigargin derivative.

57. The composition of claim 3, wherein said sesquiterpene-γ-lactone is a thapsigargin or a thapsigargin derivative.

58. The composition of claim 56, wherein said thapsigargin derivative is 8-O-(12-aminododecanoyl)-8-O-debutanoyl thapsigargin (12ADT).

59. The composition of claim 57, wherein said thapsigargin derivative is 8-O-(12-aminododecanoyl)-8-O-debutanoyl thapsigargin (12ADT).

60. (canceled)

61. (canceled)

62. The composition of claim 2, wherein said peptide comprises an amino acid sequence having a cleavage site specific for an protease selected from the group consisting of PSA, PSMA, hK2 and FAP or an enzyme having a proteolytic activity of PSA, PSMA, hK2 or FAP.

63. The composition of claim 3, wherein said peptide comprises an amino acid sequence having a cleavage site specific for an protease selected from the group consisting of PSA, PSMA, hK2 and FAP or an enzyme having a proteolytic activity of PSA, PSMA, hK2 or FAP.

64. (canceled)

65. (canceled)

66. (canceled)

67. (canceled)

68. The composition of claim 58, wherein said thapsigargin derivative is linked to the aspartic acid of a peptide having the sequence Asp-Glu*Glu*Glu*Glu (SEQ ID NO: 486), wherein at least one of the bonds designated with * is a gamma carboxy linkage.

69. The composition of claim 59, wherein said thapsigargin derivative is linked to the aspartic acid of a peptide having the sequence Asp-Glu*Glu*Glu*Glu (SEQ ID NO: 486), wherein at least one of the bonds designated with * is a gamma carboxy linkage.

70. The composition of claim 3 wherein the lecithin is selected from the group consisting of soy lecithin, egg lecithin, or a combination or a salt thereof and/or the oil is soybean oil.

71. (canceled)

72. The composition of claim 3 that additionally contains an injectable cryoprotectant, or antioxidant.

73. The composition of claim 72 wherein the cryoprotectant is sucrose and the antioxidant is EDTA or a salt thereof.

74. The composition of claim 2 wherein the cell proliferative disorder is cancer.

75. A lyophilized composition wherein upon reconstitution with water forms a pharmaceutical composition suitable for intravenous administration comprising
(a) a prodrug comprising
a therapeutically active drug comprising a sesquiterpene-γ-lactone, analogue or derivative thereof, and
a peptide comprising an amino acid sequence having a cleavage site specific for a protease associated with a cell proliferative disorder,
wherein the peptide has 20 or fewer amino acids in length,
wherein the peptide is linked to the therapeutically active drug to inhibit the therapeutic activity of the drug, and wherein the therapeutically active drug is cleaved from the peptide upon proteolysis by said protease; and
(b) a pharmaceutically acceptable vehicle;
wherein the prodrug is present in an amount of from about 1.5% to about 2.5% by weight of the total composition.

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. The composition of claim 76, wherein the lecithin-to-prodrug weight ratio is about 2.5:1 to 5:1 and/or the lecithin-to-oil weight ratio is about 10:1 to 5:2.

88. (canceled)

89. (canceled)

90. (canceled)

91. The composition of claim 16, wherein said prodrug comprises the thapsigargin derivative 8-O-(12-aminododecanoyl)-8-O-debutanoyl thapsigargin (12ADT) linked to the aspartic acid of a peptide having the sequence Asp-Glu*Glu*Glu*Glu (SEQ ID NO: 486), wherein at least one of the bonds designated with * is a gamma carboxy linkage

92. (canceled)

93. (canceled)

94. (canceled)

95. (canceled)

96. The composition of claim 90, wherein said composition may be filtered through a 0.2-micron filter and/or may be filtered through a 0.2-micron filter after a freeze-thaw stress.

97. A composition according to claim 76 wherein the pharmaceutically acceptable vehicle comprises oil droplets of having an average diameter of less than about 200 nanometers.

98. (canceled)

99. (canceled)

100. (canceled)

101. (canceled)

102. (canceled)

103. (canceled)

104. (canceled)

105. A composition according to claim 76 that exhibits a light transmittance value at 600 nm of no less than about 30%.

106. (canceled)

107. (canceled)

108. (canceled)

109. (canceled)

110. (canceled)

111. (canceled)

112. (canceled)

113. (canceled)

114. (canceled)

115. The composition of claim 75, wherein said sesquiterpene-γ-lactone is thapsigargin or a thapsigargin derivative.

116. (canceled)

117. The composition of claim 115, wherein said thapsigargin derivative is 8-O-(12-aminododecanoyl)-8-O-debutanoyl thapsigargin (12ADT).

118. (canceled)

119. (canceled)

120. (canceled)

121. The composition of claim 75, wherein said peptide comprises an amino acid sequence having a cleavage site specific for an protease selected from the group consisting of PSA, PSMA, hK2 and FAP or an enzyme having a proteolytic activity of PSA, PSMA, hK2 or FAP.

122. (canceled)

123. (canceled)

124. (canceled)

125. (canceled)

126. (canceled)

127. The composition of claim 117, wherein said thapsigargin derivative is linked to the aspartic acid of a peptide comprising the sequence of Asp-Glu*Glu*Glu*Glu (SEQ ID No: 486), wherein at least one of the bonds designated with * is a gamma carboxy linkage.

128. (canceled)

129. (canceled)

130. (canceled)

131. The composition of claim 75, wherein the lecithin is selected from the group consisting of soy lecithin, egg lecithin, or a combination or a salt thereof, the oil is soybean oil, and/or additionally, the composition contains an injectable cryoprotectant or antioxidant.

132. (canceled)

133. A process for making a pharmaceutical composition comprising:
(a) combining the composition of claim 2 with water,
(b) homogenizing the mixture to average droplet size of less than 200 nm in diameter,
(c) passing the mixture through a 02.-micron filter; and optionally, lyophilizing the filtered mixture.

134. (canceled)

135. (canceled)

136. (canceled)

137. (canceled)

138. (canceled)

139. (canceled)

140. (canceled)

141. (canceled)

142. (canceled)

143. The process of claim 133, wherein the lecithin-to-prodrug weight ratio is about 2.5:1 to 5:1, the lecithin-to-oil weight ratio is about 10:1 to 5:2, the composition may be filtered through a 0.2 micron filter after a freeze-thaw stress, the composition may be lyophilizable, the composition is chemically stable for at least 3 months, and/or the prodrug comprises the thapsigargin derivative 8-O-(12-aminododecanoyl)-8-O-debutanoyl thapsigargin (12ADT) linked to the aspartic acid of a peptide having the sequence Asp-Glu*Glu*Glu*Glu (SEQ ID NO: 486), wherein at least one of the bonds designated with * is a gamma carboxy linkage.

144. (canceled)

145. (canceled)

146. (canceled)

147. (canceled)

148. (canceled)

149. (canceled)

150. (canceled)

151. (canceled)

152. (canceled)

153. (canceled)

154. (canceled)

155. (canceled)

156. (canceled)

157. (canceled)

158. (canceled)

159. (canceled)

160. (canceled)

161. (canceled)

162. (canceled)

163. (canceled)

164. (canceled)

165. (canceled)

166. (canceled)

167. (canceled)

168. (canceled)

169. (canceled)

170. (canceled)

171. A method of treating a cell proliferative disorder in a patient comprising administering a therapeutically effective amount of the composition of claim 2 to said patient.

172. (canceled)

173. (canceled)

174. (canceled)

175. A composition for detecting and imaging a cell proliferative disorder in a subject comprising the composition of claim 2 and a phenolic linker.

176. (canceled)

177. (canceled)

178. (canceled)

179. (canceled)

180. (canceled)

181. (canceled)

182. (canceled)

183. (canceled)

184. (canceled)

185. (canceled)

186. (canceled)
